(19) United States
(12) Patent Application Publication
(10) Pub. No.: US 2025/0073384 A1
(43) Pub. Date: Mar. 6, 2025

(54) SYSTEMS, APPARATUS, AND METHODS FOR TREATING PLEURAL AND PERITONEAL SPACES

(71) Applicant: CoapTech, Inc., Baltimore, MD (US)

(72) Inventors: Steven P. TROPELLO, Baltimore, MD (US); William Alexander LeMay PATTERSON, Dundalk, MD (US); Michael Stuart ZIMMERMAN, Cockeysville, MD (US); Jared Stephen WINEBERG, Baltimore, MD (US); Olivia Kathryn Anne SKAUG, Bear, DE (US)

(73) Assignee: CoapTech, Inc., Baltimore, MD (US)

(21) Appl. No.: 18/678,208

(22) Filed: May 30, 2024

Related U.S. Application Data
(63) Continuation of application No. PCT/US2022/051714, filed on Dec. 2, 2022.
(60) Provisional application No. 63/285,850, filed on Dec. 3, 2021.

(51) Int. Cl. A61M 1/00; A61M 25/01

(52) U.S. Cl. CPC A61M 1/87; A61M 1/842; A61M 1/85; A61M 25/0108; A61M 25/0127

(57) ABSTRACT

In some embodiments, a system or apparatus includes an elongated tubular member having a first end and a second end. The elongated tubular member defines a first lumen extending from the first end to the second end, and a second lumen extending from a first port, which is defined in a sidewall of the elongated tubular member, to a second port in the second end of the elongated tubular member. An inflatable member is coupled to the elongated tubular member. The inflatable member is configured to define an echogenic volume in an inflated condition. A magnetic member is disposed within the elongated tubular member. A position of the inflatable member and the first end of the elongated tubular member within a cavity of a patient can be controllable via magnetic attraction between the magnet and an external magnetic source disposed external to the patient.

### CROSS-REFERENCE TO RELATED APPLICATIONS

This application is a continuation of International Application No. PCT/US2022/051714, filed Dec. 2, 2022, which claims priority to and the benefit of U.S. Patent Application Ser. No. 63/285,850, entitled “Systems, Apparatus, and Methods for Treating Pleural and Peritoneal Spaces,” filed Dec. 3, 2021, the disclosure of each of which is incorporated by reference herein in its entirety.

### BACKGROUND

Finite spaces within a human body, such as chest and abdominal cavities, can fill with undesirable fluid in patients suffering from pathologic conditions such as cirrhosis and pneumonia. Procedures such as thoracentesis and paracentesis can be utilized to sample and/or drain pathologic fluid from cavities of a patient. Depending on the underlying condition of the patient, however, more invasive and complicated procedures are often utilized to treat fluid collections.

Additionally, medical procedures such as thoracoscopy and laparoscopy can be performed to access chest and abdominal cavities. Such procedures, however, are often costly and require specialized providers, equipment, and designated procedural rooms. Furthermore, scopes used for such procedures often are difficult to reprocess and are often not biologically sterile.

Thus, there is a need for systems, apparatus, and methods for accessing and treating patient cavities, such as pleural and peritoneal spaces of chest and abdominal cavities, as well as particular areas within patient cavities, that reduce risks to the patient and allow for particular regions of the patient cavity to be accessed and treated quickly and easily.

### SUMMARY

In some embodiments, a system or apparatus includes an elongated tubular member having a first end and a second end. The elongated tubular member can define a first lumen extending from the first end to the second end, and a second lumen extending from a first port, which can be defined in a sidewall of the elongated tubular member, to a second port in the second end of the elongated tubular member. An echogenic member can be coupled to the elongated tubular member. A magnetic member can be disposed within the elongated tubular member, at a position adjacent to the inflatable member, and the first end of the elongated tubular member can be controllable, within a cavity of a patient, via magnetic attraction between the magnet and an external magnetic source disposed external to the patient.

### BRIEF DESCRIPTION OF THE DRAWINGS

FIG. 1 is a schematic diagram depicting a system, according to an embodiment.

FIG. 2 is a flow diagram illustrating a method of using the system depicted in FIG. 1, according to an embodiment.

FIGS. 3A-3H are schematic illustrations depicting a system in various stages of operation, according to an embodiment.

FIG. 4 is a schematic illustration depicting a system in various stages of operation, according to an embodiment.

FIGS. 5A and 5B are schematic illustrations of portions of a system including mechanical abrasion features formed as an array of projections disposed on a surface of an inflatable member, according to an embodiment.

FIGS. 6A-6C are schematic illustrations of a portion of a system including a mechanical abrasion feature including a set of ribs disposed on a surface of an inflatable member, according to an embodiment.

FIGS. 7A and 7B are schematic illustrations of a mechanical abrasion feature including a set of ribs, according to an embodiment.

FIG. 8 is a schematic illustration of an external magnetic assembly, according to an embodiment.

FIGS. 9A and 9B are schematic illustrations of an external magnetic assembly, according to an embodiment.

### DETAILED DESCRIPTION

In some embodiments, a system or apparatus includes an elongated tubular member having a first end and a second end. The elongated tubular member defines a first lumen extending from the first end to the second end, and a second lumen extending from a first port. which is defined in a sidewall of the elongated tubular member, to a second port in the second end of the elongated tubular member. An echogenic member is coupled to the elongated tubular member. A magnetic member is disposed within the elongated tubular member. A position of the echogenic member and the first end of the elongated tubular member within a cavity of a patient can be controllable via magnetic attraction between the magnet and an external magnetic source disposed external to the patient.

In some embodiments, a method includes introducing a first end of an elongated tubular member through an opening of a patient and into a cavity of the patient. An external magnetic assembly can be coupled to an external surface of the patient such that a magnetic member disposed within the elongated tubular member is urged toward the external magnetic assembly and the echogenic member contacts a surface of a wall of the cavity. The surface of the wall of the cavity and the external surface can be disposed on opposite sides of at least one tissue surface of the patient. The echogenic member can be visualized within the cavity. This technique, in which the echogenic member is urged against a surface of a wall of a body cavity, and the echogenic member and all tissue planes between the echogenic member and the external surface of the patient can be visualized by ultrasound, can be referred to as Coaptive Ultrasound (CU). The external magnetic assembly can be moved along the external surface of the patient such that the first end of the elongated tubular member is advanced within the cavity. Fluid is drawn from the cavity through the first end of the elongated tubular member, through a lumen of the elongated tubular member, and out of the second end of the elongated tubular member.

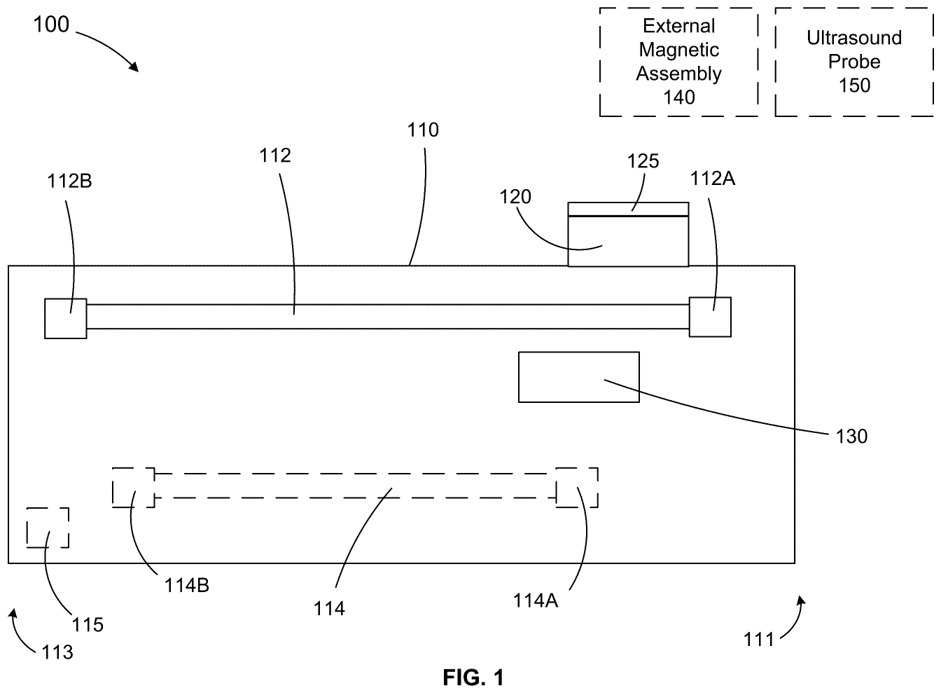

FIG. 1 is a schematic diagram depicting a system 100, according to an embodiment. As shown, the system 100 includes an elongated tubular member 110, an echogenic member 120, and a magnetic member 130. The echogenic member 120 can be coupled to the elongated tubular member 110. The magnetic member 130 can be coupled to and/or disposed within the elongated tubular member 110. In some embodiments, the system 100 can optionally include an external magnetic assembly 140 and/or an ultrasound probe 150. The elongated tubular member 110 can have a first end 111 and a second end 113 and can define a first lumen 112 extending from the first end 111 to the second end 113. The echogenic member 120 and the magnetic member 130 can be coupled to the elongated tubular member 110 near the first end 111. The first end 111 can be configured to be disposed or introduced through an opening of a subject (e.g., through an incision via, for example, an introducer assembly) and into a cavity of a patient. The cavity may include, for example, a pleural and/or peritoneal space of a chest or abdominal cavity of the patient.

The first end 111 and the second end 113 can each define at least one opening. such as a fenestration, aperture, hole, inlet, outlet, port, or the like (hereinafter generally referred to as a “port”). For example, as shown in FIG. 1, the system 100 defines a first port 112A and a second port 112B (collectively, ports 112A-B). The ports 112A-B can be configured to be in fluid communication with the first lumen 112 to enable and facilitate fluid communication through the first lumen 112. The first end 111 and/or the second end 113 can have any suitable shape. In some implementations, the first end 111 can have a conical shape or a tapered shape. In some implementations, the first end 111 can have a blunt end shape and the surface of the first end 111 of the elongated tubular member 110 can be disposed in a plane perpendicular to a central axis of a portion of the elongated tubular member 110 adjacent the first end 111. In some implementations, the first end 111 can include a sharp edge.

In some implementations, the second port 112B can be configured to be coupled to a source of fluid (not shown) (e.g., via any suitable connection component(s)) such that fluid can be received into the first lumen 112 from the source of fluid via the second port 112B. In some implementations, the port 112B can be configured to be coupled to a source of suction (not shown) (e.g., via any suitable connection component(s)) such that fluid can be drawn through the first port 112A, through the first lumen 112, and through the second port 112B. Thus, the second port 112B can be configured to function as an inlet and/or an outlet. In some implementations, the second port 112B can be configured to interface with (e.g., be coupled to) a fluid conveying device (not shown) such as a pump (e.g., a peristaltic pump) such that fluid conveyed by the fluid conveying device can be received into the first lumen 112 for delivery to the patient from a source of fluid via the second port 112B. In some implementations, the second port 112B can include and/or be coupled to any suitable fluid connector or valve.

In some implementations, the elongated tubular member 110 can define one or more additional lumens. For example, the elongated tubular member 110 can optionally define a second lumen 114 extending between the first end 111 and the second end 113. For example, the second lumen 114 can extend along a length (e.g., a partial length) of the elongated tubular member 110, such as from a first port 114A defined in a sidewall of the elongated tubular member 110 at a location spaced from the first end 111 (e.g., between the first end 111 and the second end 113) to a second port 114B defined in the second end 113 of the elongated tubular member 110 or in a sidewall of the elongated tubular member 110 at a location adjacent to the second end 113. The first port 114A and the second port 114B can include one or more fenestration(s), aperture(s), or hole(s). In some implementations, the second port 114B can include and/or be coupled to any suitable fluid connector or valve. The first port 114A and the second port 114B can be configured to be in fluid communication with the second lumen 114 to enable and facilitate fluid communication through the second lumen 114. In such implementations, fluid can be drained or evacuated from a cavity of the patient via the first port 114A, the second lumen 114, and the second port 114B. In some implementations, the elongated tubular member 110 can instead be an elongated tubular assembly including multiple elongated tubular members (also referred to as tubes) each defining any suitable number of lumens and/or openings, such as any of the lumens and/or openings described herein. In some implementations, the elongated tubular member or assembly can include any suitable number of tubular portions or segments coupled to an adjacent tubular portion or segment via any suitable connector (e.g., a fluid connector). In some implementations, one or more elongated tubular members included in an elongated tubular assembly can be disposed coaxially relative to one or more other elongated tubular members of the elongated tubular assembly. In some implementations, an elongated tubular member included in the elongated tubular assembly can be formed as an outer tubing within which one or more remaining elongated tubular members can be disposed.

The echogenic member 120 can be coupled to the elongated tubular member 110 at or near the first end 111 of the elongated tubular member 110. In some embodiments, the echogenic member 120 can be implemented as an inflatable member, which can be configured to define an echogenic volume (e.g., in an inflated condition of the inflatable member). The echogenic volume can be configured to be visualized via ultrasound, such that the location of the inflatable member (and, thus, the first end 111 of the elongated tubular member 110) within a patient can be verified. For example, the inflatable member can be configured to transition between an uninflated configuration and an inflated configuration. The inflatable member can be configured to receive a fluid (e.g., gas, liquid, or an otherwise flowable substance or material) within an interior of the inflatable member such that the inflatable member transitions between the uninflated and the inflated configuration. The fluid can be, for example, an echogenic fluid (e.g., water and/or saline).

The inflatable member can be fluidly coupled to an inflation lumen (not shown) for fluid communication with a source of fluid. In some implementations, an inflation lumen can be at least partially disposed in or defined by the elongated tubular member 110. In some implementations, the inflation lumen can include an inflation port on or near the second end 113 of the elongated tubular member 110) and the inflation lumen can be in fluid communication with an interior of the inflatable member via one or more openings in a sidewall of the elongated tubular member 110 such that the inflatable member can be transitioned between an inflated and an uninflated configuration via fluid traveling through the inflation port and the inflation lumen. In some implementations, a central axis of the inflation lumen can extend substantially parallel to a central axis of the elongated tubular member 110.

In some implementations, the inflation lumen can be separate from the elongated tubular member 110. For example, the inflation lumen can be defined by a second tubular member (not shown) that can be disposed external to or within a lumen of the elongated tubular member 110. For example, the inflatable member can be filled and/or inflated with a fluid and/or contrast medium such that the inflatable member defines an echogenic space or volume detectable using ultrasound imaging. In some implementations, the inflatable member can be filled and/or inflated with contrast medium such that the location of the inflatable member can be visualized using fluoroscopy. Inflation of the inflatable member can increase the target size of the inflatable member for visualization. In some implementations, for example, a source of echogenic fluid can be fluidly coupled to an interior of the inflatable member via an inflation lumen of the elongated tubular member 110 such that the inflatable member can transition between an uninflated and an inflated configuration via providing the echogenic fluid from the source of echogenic fluid to the interior of the inflatable member via the inflation lumen.

In some implementations, the inflatable member can be configured to form, in an inflated and/or uninflated configuration, any suitable shape and/or any suitable size. For example, the inflatable member or an outer perimeter of the inflatable member can be elliptical, spherical, cylindrical, rectangular, tear drop, or any other suitable shape. In some implementations, the shape of the inflatable member can be chosen based on a particular application of the system 100. In some implementations, the inflatable member can have any suitable material properties, wall thicknesses, and/or inflated outermost diameters. In some implementations, the inflatable member can be formed of any suitable material such as, for example, a low durometer urethane, polyurethane, silicone, and/or polyvinyl chloride (PVC). In some implementations, the material forming the inflatable member can be based on a particular application of the system 100. In some implementations, the inflatable member can be formed of or include an echogenic material or coating. In some implementations, the shape and/or material of the inflatable member may be selected to improve ultrasound visualization in particular regions of a patient's body.

In some implementations, the echogenic member 120 can surround the elongated tubular member 110, for example, as an inflatable member in an inflated and/or an uninflated configuration. For example, the echogenic member 120 can surround the elongated tubular member 110) about the first end 111 when coupled to the elongated tubular member 110 at or near the first end 111. In some implementations, the inflatable member can be configured to extend laterally from the first end 111 of the elongated tubular member 110 in an inflated and/or uninflated configuration. In some implementations, the inflatable member can be configured to extend distally from the first end 111 of the elongated tube 112 in an inflated and/or uninflated configuration. In some implementations, the inflatable member can be disposed in or on the elongated tubular member 110 such that a portion of the elongated tubular member 110 extends distally of the inflatable member when the inflatable member is in an inflated and/or uninflated configuration. In some implementations, the inflatable member can have two ends (e.g., cuffs). and each end can be sealed to an outer surface of the elongated tubular member 110. For example, the inflatable member can be or include an elastic or flexible bag or sac. In some implementations, the inflatable member can include a balloon.

In other embodiments, echogenic member 120 can be implemented as a component having echogenic properties, such as having a textured surface (e.g., including dimples and/or roughness) for redirecting scattered waves back toward the ultrasound probe 150. For example, in some embodiments, the elongated tubular member 110 or another components of the system 100 can include at least a portion having a textured surface and/or formed of an echogenic material for increased echogenicity such that an inflatable member is not needed for ultrasound visualization of a portion of the system 100 within a body cavity (e.g., a first or distal end of the elongated tubular member 110). The echogenic member 120 need only provide an acoustic impedance that differs from that of the tissue against which the echogenic member is coapted, e.g., by magnetic attraction between magnetic member 130 and external magnetic assembly 140, producing reflection of some of the incident ultrasonic energy from ultrasound probe 150.

In some embodiments, the system 100 can include or be coupled to one or more mechanical abrasion features 125 (also referred to as “mechanical abrasion elements” or “mechanical abrasion portions”). The mechanical abrasion feature(s) 125 can be disposed on or coupled to the echogenic member 120 (e.g., an outer surface of the echogenic member 120). The mechanical abrasion feature(s) 125 can be placed in abrasive contact with unwanted tissue and/or thick fluid and moved (e.g., rotated and/or translated) to break down the unwanted tissue and/or thick fluid using friction between the mechanical abrasion feature(s) 125 and the unwanted tissue and/or thick fluid. In some embodiments, the mechanical abrasion feature(s) 125 can be used to perform mechanical tissue lysis and/or debridement.

In some implementations, the mechanical abrasion feature(s) 125 can be formed as an array of protrusions disposed on and extending from an inflatable member (e.g., a balloon). The inflatable member can be, for example, the echogenic member 120 implemented as an inflatable member as described above. The array of protrusions can include bumps, ridges, and/or any other suitable shapes. In some embodiments, each protrusion can include a textured surface to increase frictional contact between the protrusion and unwanted tissue and/or fluid. For example, the surface of each protrusion may be more textured than the surface of the inflatable member on which the protrusion are disposed.

In some implementations, the mechanical abrasion feature 125 can be formed as a set of ribs extending along an outer surface of an inflatable member (e.g., a balloon). The inflatable member can be, for example, the echogenic member 120 implemented as an inflatable member as described above. In some implementations, the inflatable member can include an inflatable member not used for ultrasound visualization. Each rib of the set of ribs can extend from a first end to a second end of the inflatable member (e.g., from a first location adjacent a first portion of the elongated tubular member 110 to a second location adjacent a second portion of the elongated tubular member 110). In some implementations, each rib of the set of ribs can be disposed in a plane including a central axis of the inflatable member and/or the elongated tubular member 110. In some implementations, the mechanical abrasion feature 125 can include a first end connector and a second end connector and each rib of the set of ribs can include a first end coupled to the first end connector and a second end coupled to the second end connector. The first end connector and the second end connector can each have a circular shape defining a through-hole configured to receive a portion of the elongated tubular member.

In some implementations, the set of ribs can be transitioned from an initial configuration (e.g., for insertion into a body cavity) to an expanded configuration by expanding the inflatable member (e.g., from a collapsed configuration to a substantially spheroidal or spherical configuration). In some implementations, when transitioning from the initial configuration to the expanded configuration, the ribs can transition from a substantially straight shape to a curved shape or from a first curved shape to a second curved shape having a larger radius of curvature than the first curved shape. The set of ribs can be arranged to define openings between adjacent ribs. In some embodiments, the ribs can be echogenic and/or can allow the inflatable member to be visualized through the openings defined between adjacent ribs. In some implementations, each rib of the set of ribs can have a textured surface with sufficient roughness such that the ribs can be used to break down unwanted tissue and/or thick fluid using frictional contract between the rib and the unwanted tissue and/or thick fluid (e.g., while translating and/or rotating the set of ribs). In some implementations, each rib can include one or more sharp edges configured to contact and break down unwanted tissue and/or thick fluid.

In some embodiments, the mechanical abrasion feature 125 (e.g., the set of ribs and/or end connectors) can be magnetic or can include magnetic portions arranged such that the mechanical abrasion feature 125 can be translated and/or rotated within a body cavity due to magnetic interaction with the external magnetic assembly 140. In some embodiments, the mechanical abrasion feature 125 can be configured to rotate relative to the elongated tubular member 110) and/or the inflatable member (e.g., due to urging due to magnetic interaction). When the mechanical abrasion feature(s) 125 are rotatable relative to the elongated tubular member 110 and/or the inflatable member, the external magnetic assembly 140 can cause the mechanical abrasion feature 125 to rotate via rotation of one or more magnets of the external magnetic assembly 140 to urge magnetic portion(s) included in or coupled to the mechanical abrasion feature(s) 125 to rotate.

In some embodiments, the mechanical abrasion feature 125 can be fixed relative to the elongated tubular member 110 and/or the inflatable member such that rotation of the mechanical abrasion feature 125 causes rotation of the inflatable member and at least the first end 111 of the elongated tubular member 110. In some embodiments, the magnetic member 130 can be fixed relative to the mechanical abrasion feature 125 (e.g., via being directly coupled or indirectly coupled via other components such as the elongated tubular member 110 and/or the inflatable member) and the external magnetic assembly 140 can magnetically interact with magnetic member 130 such that the mechanical abrasion feature 125 can be translated and/or rotated due to the external magnetic assembly 140 urging (e.g., rotating and/or translating) the magnetic member 130.

In some implementations, the mechanical abrasion feature 125 can be rotated under the control of the external magnetic assembly 140 (e.g., via rotation of one or more magnets of the external magnetic assembly 140) while the mechanical abrasion feature 125 are disposed in a body cavity to apply abrasion to tissue and/or fluid within the body cavity. In some implementations, such as when the mechanical abrasion feature 125 is fixed relative to the elongated tubular member 110, rotation of the mechanical abrasion feature 125 in a first direction can cause the first end 111 to rotate relative to the second end 112 of the elongated tubular member 110. When the external magnetic assembly 140 no longer applies an urging magnetic force to the mechanical abrasion feature 125 and/or the magnetic member 130 (e.g., due to being removed from a surface of the patient), the first end 111 of the elongated tubular member 110 can unwind relative to the second end 112 to rotate the mechanical abrasion feature 125 in a second direction opposite the first direction to apply additional abrasion to tissue and/or fluid. In some implementations, such as when the mechanical abrasion feature 125 are fixed relative to the elongated tubular member 110, the elongated tubular member 110 can be rotated (e.g., via grasping and rotating the second end 112 or another portion of the elongated tubular member 110 disposed outside the patient's body) to cause rotation of the mechanical abrasion feature 125 within the body cavity (e.g., in a first direction and/or sequentially and repeatedly in a first direction and then an opposite second direction).

The magnetic member 130 can be disposed within the elongated tubular member 110. The magnetic member 130 can be configured such that a position of the echogenic member 120 and the first end 111 of the elongated tubular member 110 (e.g., when disposed at least partially within a cavity of a patient) can be controlled at least in part via magnetic attraction or interaction between the magnetic member 130 and an external magnetic source (e.g., the external magnetic assembly 140) disposed external to the patient. In some implementations, the echogenic member 120 can be disposed at a position situated closer to the first end 111 of the elongated tubular member 110 than the magnetic member 130.

In some implementations, the magnetic member 130 can be coupled to the elongated tubular member 110 at or near the first end 111. The magnetic member 130 can be configured such that movement of the magnetic member 130 causes corresponding movement of the first end 111 of the elongated tubular member 110. In some implementations, the magnetic member 130 can be coupled directly to the elongated tubular member 110. In some implementations, the magnetic member 130 can be embedded within a sidewall of the elongated tubular member 110. In some implementations, the magnetic member 130 can be disposed within the echogenic member 120 (e.g., when implemented as an inflatable member as described above), and/or at least partially surrounded or encompassed by the inflatable member. In some implementations, the magnetic member 130 can be coupled directly to the inflatable member.

The magnetic member 130 can be or include, for example, any suitable magnet or magnetic member (e.g., a magnet, electromagnet, etc.). In some implementations, the magnetic member 130 can include a permanent magnet such as a rare earth magnetic (formed of rare earth materials, including neodymium iron boron (NdFeB) or samarium cobalt (SmCo)), an aluminum nickel cobalt (AlNiCo) magnet, a ceramic/ferrite magnet, and/or any other suitable permanent magnet. In some implementations, the magnetic member 130 can include a temporary magnet. In some implementations, the magnetic member 130 can be an electromagnet, such as a solenoid (with or without a solid core). In some implementations, the magnetic member 130 can generate a magnetic field having an orientation (i.e., north (N) and south (S) poles). In other implementations, the magnetic member 130 can be formed of a ferromagnetic material that is not magnetized, i.e., does not generate its own magnetic field, but can be affected by an externally-applied magnetic field. For example, the magnetic member 130 can be formed of iron, and application of an external magnetic field can attract the iron toward the source of the field, applying a force to the magnetic member 130. The magnetic member 130 can have any suitable shape. For example, the magnetic member 130 can be shaped in the form of an elongated rectangle. As another example, the magnetic member 130 can be shaped in the form of a cylinder. As yet another example, the magnetic member 130 can be generally arcuate in shape. As another example, the magnetic member 130 can be shaped as a circular disk.

Although FIG. 1 shows only one magnetic member 130, the system 100 can include any suitable number of magnetic members that can be the same or similar in structure and/or function to the magnetic member 130. For example, the system 100 can include two, three, four, five or more magnetic members. The magnetic members can be arranged in any suitable arrangement such that the first end 111 of the elongated tubular member 110 can be urged toward and/or away from an external magnetic assembly (e.g., the external magnetic assembly 140) due to the interaction between the magnetic members and the external magnetic assembly. For example, the magnetic members can be arranged in a line or row within a portion of the elongated tubular member 110. In some implementations, the magnetic member 130 can be a first magnetic member, and the system 100 can include a second magnetic member disposed within the elongated tubular member 110 adjacent to or spaced from the first magnetic member 130.

The external magnetic assembly 140 can include any suitable magnet or magnetic material configured to apply a magnetic field to a region encompassing at least a portion of a subject, such as a patient's body. The magnetic field applied by the external magnetic assembly 140 can be configured to, for example, interact with the magnetic field generated by the magnetic member 130, such as to generate a force (e.g., an electromagnetic force) on the magnetic member 130. The external magnetic assembly 140 can include, for example, a permanent magnet, such as any of the magnet types described above. In some implementations, the external magnetic assembly 140 can be an electromagnet, such as a solenoid. In some implementations, the external magnetic assembly 140 can generate a magnetic field having an orientation (i.e., north (N) and south (S) poles). In some implementations, the magnetic member 130 and/or external magnetic assembly 140 can be formed of a ferromagnetic material that is not magnetized, i.e. does not generate its own magnetic field, but can be affected by an externally-applied magnetic field. For example, the external magnetic assembly 140 and/or the magnetic member 130 can be formed of iron or steel, and application of an external magnetic field can attract the iron toward the source of the field, applying a force to the external magnetic assembly 140 and/or the magnetic member 130.

Moreover, the external magnetic assembly 140 can be any suitable external magnetic assembly configured to urge or attract the magnetic member 130 (e.g., via magnetic attraction) toward the external magnetic assembly 140 through patient tissue (e.g., through the skin, intervening tissue, and a cavity lining of the patient). For example, in some implementations, the external magnetic assembly 140 can include a handle. In some implementations, the external magnetic assembly 140 can include one magnetic element configured for magnetic interaction with the magnetic member 130. In some implementations, the external magnetic assembly 140 can include any suitable number of magnetic elements (e.g., two magnetic elements) configured for magnetic interaction with one or more magnetic members 130 of the elongated tubular member 110. In some implementations, the external magnetic assembly 140 can include a number of magnetic elements corresponding to (e.g., equal to) the number of magnetic members 130 disposed within or coupled to the elongated tubular member 110 (e.g., two magnetic elements and two magnetic members 130). In some implementations, the external magnetic assembly 140 can include a housing configured to support one or more magnetic elements and/or define a space within which one or more magnetic elements can be disposed. The housing (e.g., one or more surfaces of the housing) can be configured to contact a surface of a subject as described herein and, in some implementations, the one or more magnetic elements can be separated from the subject via one or more housing portions when the external magnetic assembly 140 is disposed in contact with a surface of the subject. In some implementations, the housing can maintain the one or more magnetic elements at a particular distance from the surface of the subject when the external magnetic assembly 140 is disposed in contact with the surface of the subject.

In some implementations, the external magnetic assembly 140 can be configured to be disposed on or adjacent to (e.g., in contact with) a surface of a subject, such as on a patient's skin. For example, the external magnetic assembly 140 can be configured to apply a magnetic field to at least a portion of a patient's body that can interact with the magnetic field generated by the magnetic member 130 through various body tissue and organs disposed between the external magnetic assembly 140 on the patient's skin, the magnetic member 130 within the patient's body, and across any suitable distance (e.g., about 1 cm, about 2 cm, about 4 cm, about 6 cm, about 8 cm, about 10 cm, about 15 cm, and/or about 20 cm). For example, the external magnetic assembly 140 can be configured to be disposed at a cutaneous surface of a patient, such as in a region of the chest or abdomen of the patient. The interaction between the magnetic field generated by the external magnetic assembly 140 and the magnetic field generated by the magnetic member 130 can produce a magnetic force that urges the position of the magnetic member 130 in a direction toward or away from the external magnetic assembly 140, to thereby urge and/or move the first end 111 of the elongated tubular member 110 in a direction relative to the external magnetic assembly 140. In some implementations, the external magnetic assembly 140 can be configured to change the position of (e.g., translate) the first end 111 of the elongated tubular member 110 in the direction of movement of the external magnetic assembly 140 (e.g., along a longitudinal or central axis of a portion of the elongated tubular member 110 adjacent the first end 111). In some implementations, the external magnetic assembly 140 can have a first side (not depicted) and a second side (not depicted), the second side disposed on a side of the external magnetic assembly 140 opposite that of the first side. The external magnetic assembly 140 can have a first pole oriented in the direction the first side faces and a second pole oriented in the direction the second side faces, the first pole having an opposite polarity to the second pole.

In some implementations, the external magnetic assembly 140 can be configured to attract the magnetic member 130 toward the external magnetic assembly 140 (e.g., if the external magnetic assembly 140 is disposed near the magnetic member 130 with a first pole applying a magnetic force to the magnetic member 130 of an opposite polarity as the magnetic member 130 facing the external magnetic assembly 140). In some implementations, the external magnetic assembly 140 can be configured to repel the magnetic member 130 away from the external magnetic assembly 140 (e.g., if the external magnetic assembly 140 is disposed near the magnetic member 130 with a first pole applying a magnetic force to the magnetic member 130 of the same polarity as the magnetic member 130 facing the external magnetic assembly 140).

In some implementations, the external magnetic assembly 140 can include one or more rotatable magnets. The one or more rotatable magnets can be arranged to rotate relative to a bottom surface of the external magnetic assembly 140 such that, when the bottom surface of the external magnetic assembly 140 is disposed on a surface of a patient, rotation of the one or more rotatable magnets can cause one or more magnets (e.g., the magnetic member 130) and/or the magnetic mechanical abrasion feature(s) 125) of the system 100 disposed within a body cavity of the patient to rotate. In some implementations, the external magnetic assembly 140 can include, for example, a hand crank coupled to the one or more rotatable magnets such that rotation of the hand crank causes rotation of the one or more rotatable magnets in a first direction and/or a second direction opposite the first direction. In some implementations, the external magnetic assembly 140 can include, for example, a motor assembly coupled to the one or more rotatable magnets and configured to rotate the one or more rotatable magnets in a first direction and/or a second direction opposite the first direction (e.g., in response to interaction with an activation element such as a button, switch, or foot pedal).

In some implementations, the ultrasound probe 150 can be any suitable ultrasound probe configured for visualization of the echogenic member 120 within the patient. An image may be generated (e.g., on a screen of a computer) such that the echogenic member 120, or an interior of the echogenic member 120 (e.g., when implemented as an inflatable member as described above), can be observed by a user.

In some implementations, the elongated tubular member 110 can optionally include a coupling member 115 configured to be operatively coupled to an ultrasonic pulse generator, such that the system 100 can deliver ultrasonic pulses generated by the ultrasonic pulse generator to a location within the cavity. For example, pulse delivery mechanism(s) such as one or more piezoelectric transducers can be coupled to (e.g., embedded within) the first end 111 of the elongated tubular member 110. The piezoelectric transducers can be coupled to the coupling member 115 (and thus can be coupleable to the ultrasonic pulse generator) via any suitable electronic coupling, such as via one or more wires or leads extending from the piezoelectric transducers to the coupling member 115 within the elongated tubular member 110. When the piezoelectric transducers are operatively coupled to the ultrasonic pulse generator and disposed within the cavity, the ultrasonic pulse generator can provide an electrical signal to the piezoelectric transducers, causing the piezoelectric transducers to generate an ultrasonic pulse that propagates through the cavity. The ultrasonic pulse propagation can cause the contents of the cavity (e.g., thick fluid) to break down. In some implementations, the coupling member 115 can be or include a vibration conducting coupling, interface, or the like.

The system 100 can include various functionality. For example, the elongated tubular member 110 can be formed of any suitable material (e.g., polyurethanes, urethanes, silicone composites, various polymers, such as thermoplastics and/or thermoset plastics), which may be chosen based on a particular application or medical procedure in which the system 100 is implemented. The material for forming the elongated tubular member 110 can be flexible and/or impart flexibility to the elongated tubular member 110 and may be biologically inert. In some implementations, at least a portion of an outer surface of the elongated tubular member 110 can be rough and/or abrasive such that the outer surface of the elongated tubular member 110 can be used to facilitate an inflammatory or debridement process within the cavity. Additionally, the elongated tubular member 110 can have any suitable geometric shape and/or any suitable length. In some embodiments, the elongated tubular member 110 can define any suitable number of lumens that can include and/or receive any suitable number or type of conduits or tubes to be used for any suitable medical procedure. For example, in some embodiments, the elongated tubular member 110 can have a smaller diameter and only the first lumen 112 and may be used only to drain fluid from a cavity. In some embodiments, the elongated tubular member 110 can have a larger diameter and a number of lumens (e.g., for draining complex fluid collections from a cavity, irrigating a cavity, and/or performing another operation or procedure).

As described above, in some implementations, alternatively or additionally to including the echogenic member 120 (e.g., an inflatable member), the elongated tubular member 110 can include or be formed, at least in part, of an echogenic material such that the elongated tubular member 110 can be imaged (e.g., via ultrasound visualization) when the first end 111 of the elongated tubular member 110 is disposed within a body cavity. For example. an echogenic material can be imbedded along a length of the elongated tubular member 110, such as along a portion of the length of the elongated tubular member 110 near or adjacent to the first end 111. As another example, the elongated tubular member 110 can, alternatively or in addition, be formed of an echogenic material along at least a portion of a length of the elongated tubular member 110, such as near or adjacent to the first end 111.

As another example, the second port 112B and/or the second port 114B can include any suitable auxiliary or interfacing component. For example, in some implementations, the second port 112B and/or the second port 114B can include or be coupled to a gauge, a sensor and/or transducer such as a flow meter, a flow control device, a flow regulator, or the like. As another example, in some implementations, the second port 112B and/or the second port 114B can include or be configured to interface with or be coupled to any suitable medical device, such a closure device.

In some implementations, although not shown, the system 100 can include a light source and/or image capture device (e.g., a camera). For example, a light source and/or image capture device (e.g., a camera) can be attached to the elongated tubular member 110, such as at or projecting from the first end 111, to facilitate visualization during use of the system 100.

In some embodiments, the system 100 can be used in any suitable clinical and/or medical applications. For example, the system 100 can be used for acute and chronic diagnostic and therapeutic drainage, pleurodesis, and/or medical or surgical debridement. For example, in some applications such as those involving acute and chronic diagnostic and therapeutic drainage, the elongated tubular member 110 can be used as a specialized conduit for draining fluid from a chest cavity (e.g., thoracentesis) and/or an abdominal cavity (e.g., paracentesis). The first end 111 of the elongated tubular member 110 can be inserted and directed using coaptive ultrasound to regions of a cavity with significant fluid to increase the volume of fluid removal. In some implementations, the elongated tubular member 110 can be left in place for temporary drainage of the cavity. In other applications, such as for treating malignant pleural effusions (MPE), an indwelling tunneled pleural catheter (TPC) can be placed permanently and permit the patient to self-drain at home. For example, the elongated tubular member 110 can be a TPC or can be formed to perform the functions of a TPC. For such implementations, the elongated tubular member 110 can have specific dimensions or be made from a specific material to allow the elongated tubular member 110 to function as a TPC and remain in place relative to the patient for an extended period of time.

In some implementations, the elongated tubular member 110 can be used for pleurodesis by delivering a talc poudrage or slurry (e.g., via the first lumen 112 or the second lumen 114). The talc poudrage or slurry can be used to freeze the lung to the chest wall. In some implementations, to perform medical or surgical debridement, the elongated tubular member 110 can be used for debridement (e.g., via engaging a sharpened first end 111 and/or an external textured surface of the elongated tubular member 110 with fibrinous septations and/or moving the first end 111 relative to fibrinous septations). Furthermore, the elongated tubular member 110 can be used to deliver medications (e.g., DNAse, TPA and antibiotics) to a patient cavity to help clean out the cavity. The elongated tubular member 110 can also be used to irrigate the cavity and/or perform ultrasonic fibrinolysis within the cavity.

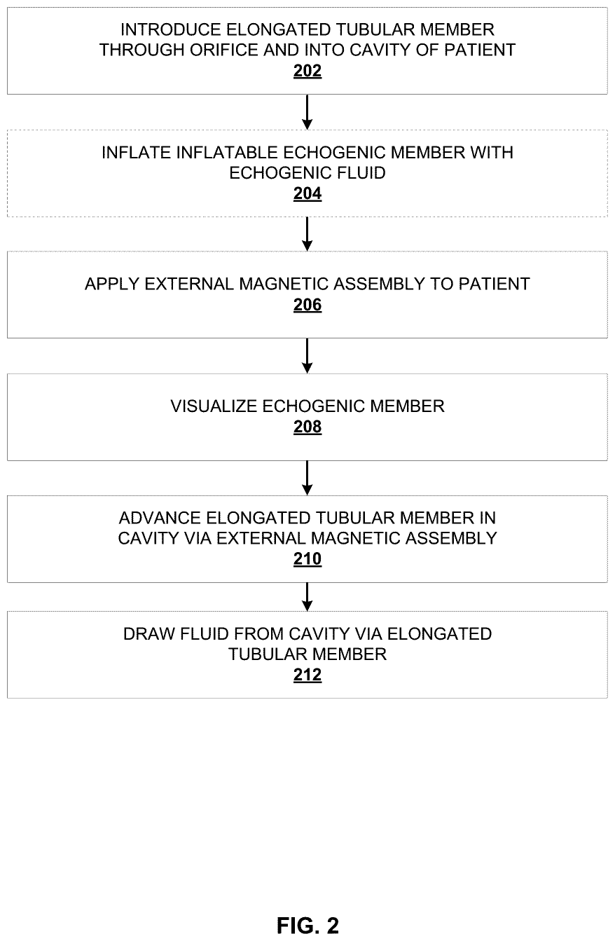

FIG. 2 is a flow diagram illustrating a method 200 of using a system such as the system 100, according to an embodiment. The method 200 can be implemented, for example, using any system or device as described herein, such as the system 100.

As shown at 202, the method 200 can include introducing a first end of an elongated tubular member through an opening of a patient and into a cavity of the patient. In some implementations, the cavity can be a pleural cavity. In some implementations, introducing the first end of the elongated tubular member through the opening of the patient can include, for example, inserting a first end of a guidewire through the opening and into the cavity of the patient, where the first end of the elongated tubular member is introduced through the opening and into the cavity of the patient via being advanced over the guidewire.

Optionally at 204, in embodiments in which the echogenic member coupled to the elongated tubular member is an inflatable member, the inflatable member can be inflated with echogenic fluid such that the inflatable member expands from an uninflated configuration to an inflated configuration. At 206, an external magnetic assembly can be applied to an external surface (e.g., a cutaneous surface) of the patient such that a magnetic member disposed within the elongated tubular member is urged toward the external magnetic assembly and the inflatable member contacts a surface of a wall of the cavity, the surface of the wall of the cavity and the external surface disposed on opposite sides of at least one tissue surface of the patient.

At 208, the echogenic member can be visualized within the cavity. In some implementations, visualizing the echogenic member within the cavity can include, for example, visualization using an ultrasound probe applied to the external surface of the patient.

At 210, the external magnetic assembly can be moved along the external surface of the patient such that the first end of the elongated tubular member is advanced within the cavity. In some implementations, moving the external magnetic assembly along the external surface of the patient such that the first end of the elongated tubular member is advanced within the cavity can include, for example, moving the external magnetic assembly such that the first end of the elongated tubular member is advanced to break up fibrinous septations.

At 212. fluid can be drawn from the cavity through the first end of the elongated tubular member, through a lumen of the elongated tubular member, and out of a second end of the elongated tubular member. In some implementations, the fluid drawn from the cavity through the first end of the elongated tubular member can be a first fluid. The first fluid can include, for example, a bodily fluid (e.g., a pathologic fluid). In some implementations, a second fluid can be introduced to the cavity of the patient via the second end of the elongated tubular member, the lumen of the elongated tubular member, and the first end of the elongated tubular member. In some implementations, the second fluid can include, for example, an irrigation fluid, a medical substance, a medication, or the like.

In some implementations, the fluid drawn from the cavity through the first end of the elongated tubular member can be a first fluid and the lumen can be a first lumen. A second fluid can be introduced to the cavity of the patient via a first port, a second lumen, and a second port defined by the elongated tubular member, the second lumen extending from the first port to the second port. The second fluid can be or include, for example, an irrigation fluid, a medical substance, a medication, or the like. The second fluid can be introduced to the cavity and the first fluid can be drawn from the cavity simultaneously.

In some implementations, the method 200 can optionally include rotating or translating a mechanical abrasion feature(s) such as the mechanical abrasion feature(s) 125 as described above. For example, the mechanical abrasion feature 125 can be rotated (e.g., continually) and/or translated due to a magnetic interaction with the external magnetic assembly 140 disposed outside of the patient. In some implementations, the mechanical abrasion feature 125 can rotate relative to either the elongated tubular member 110 or the echogenic member 120. In some implementations, the mechanical abrasion feature 125 can be rotated and/or translated by manually rotating and/or translating a proximal end of the elongated tubular member 110 disposed outside of the patient.

In some implementations, the method 200 can further include, for example, delivering one or more ultrasonic pulses to a location within the cavity via an ultrasonic pulse generator coupled to a coupling member of the elongated tubular member.

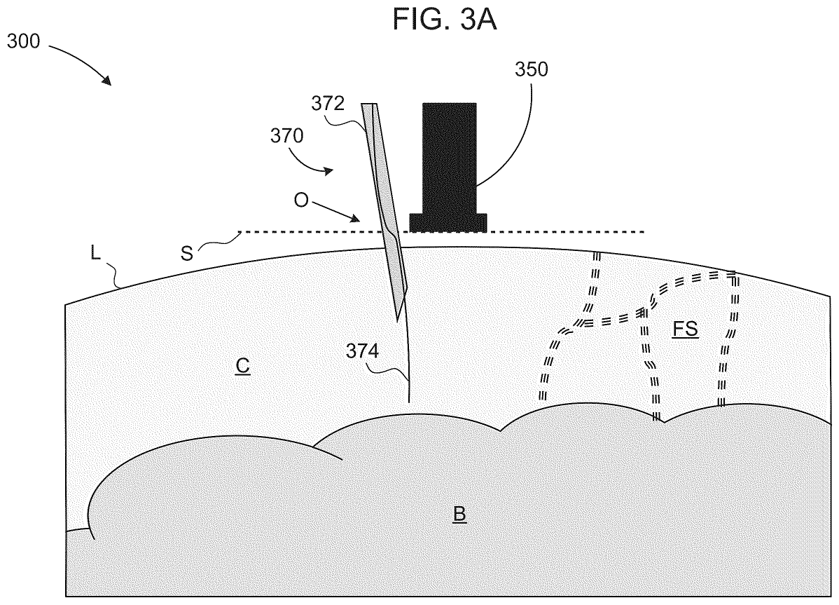

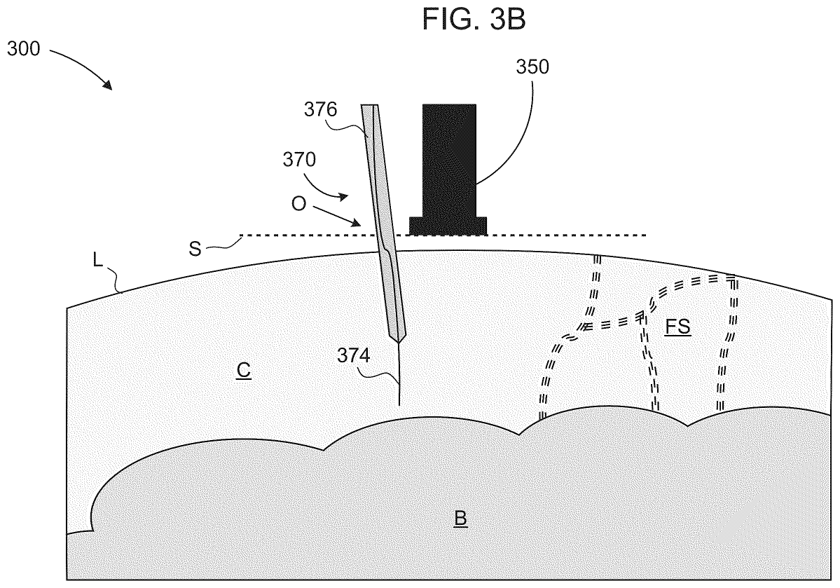

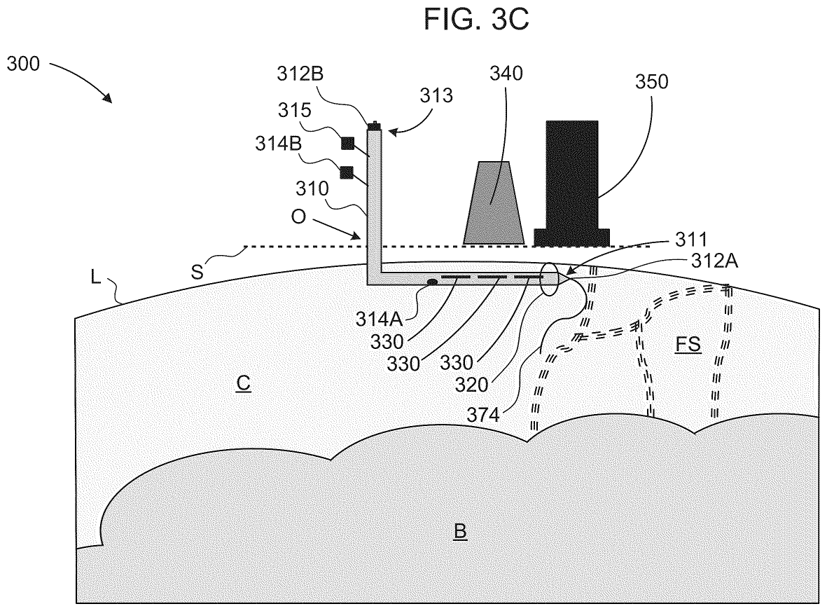

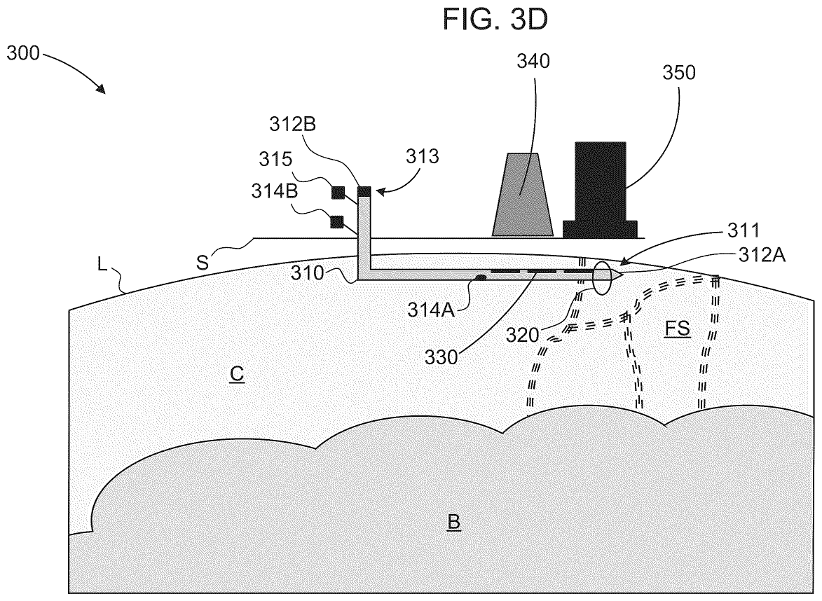

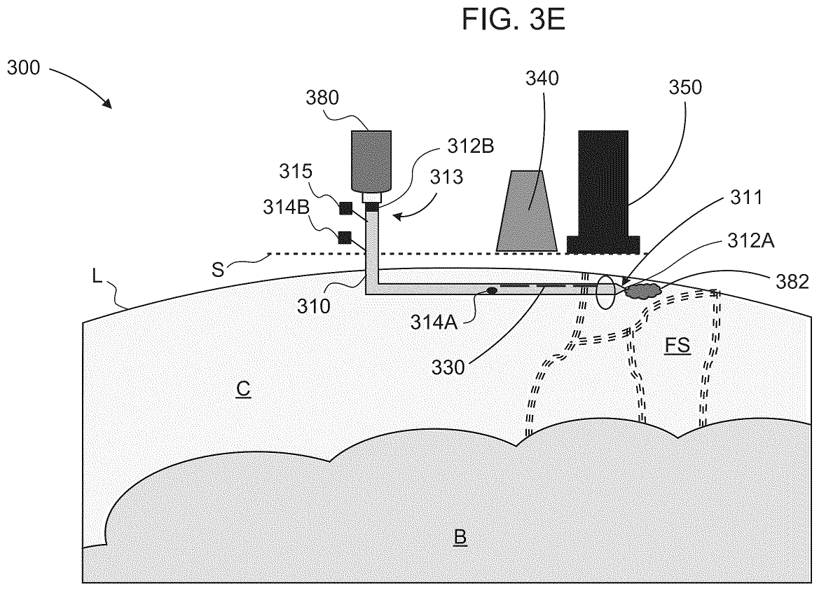

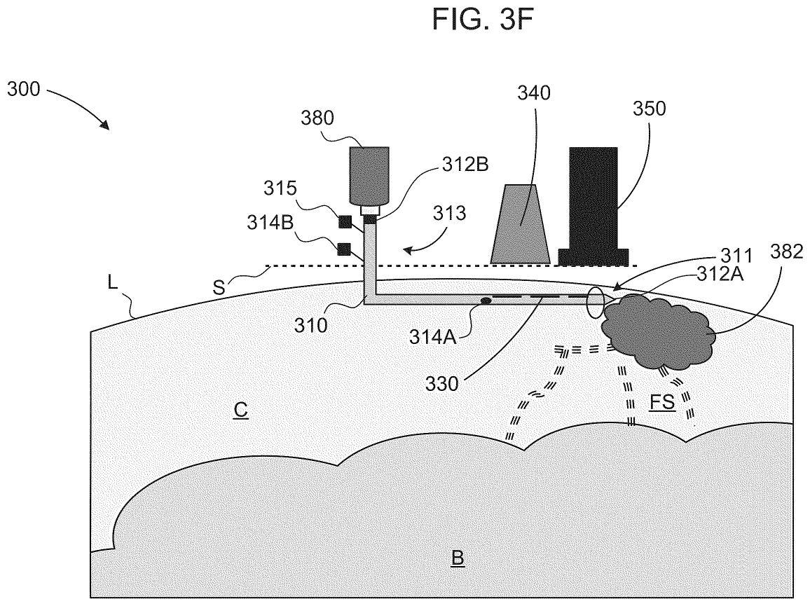

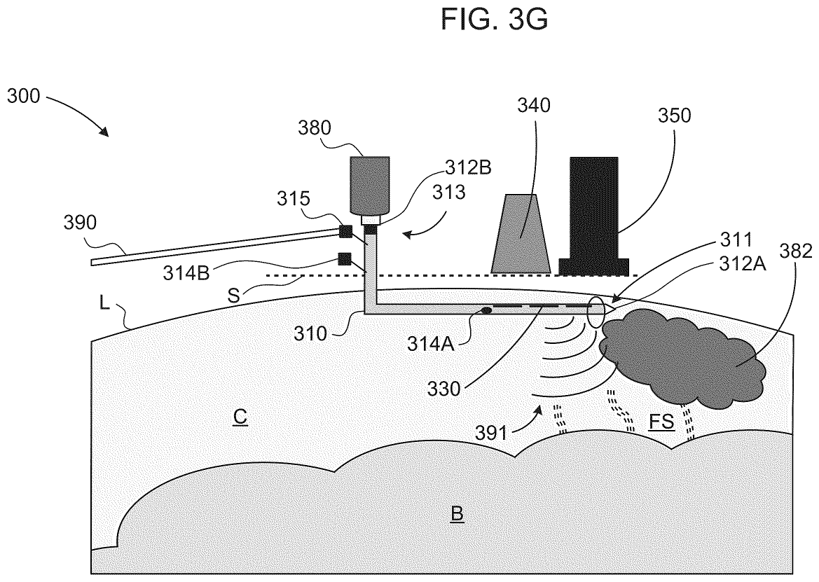

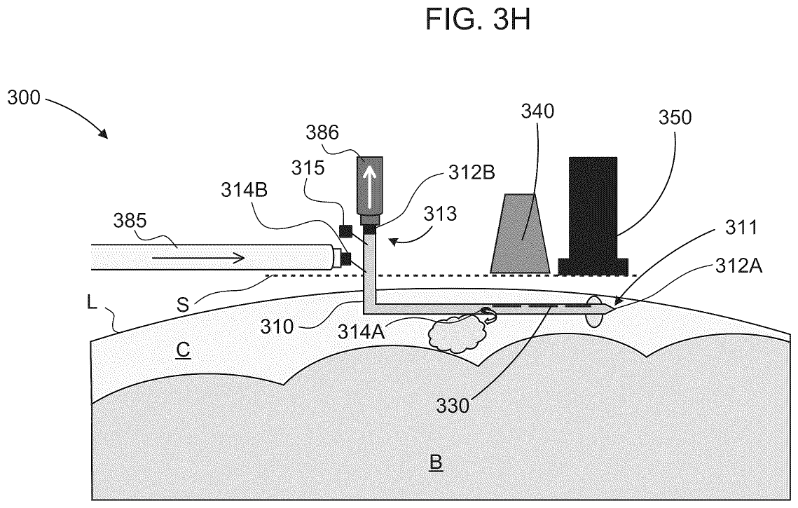

FIGS. 3A-3H are schematic illustrations of a system 300 in various stages of operation, according to an embodiment. The system 300 can be, for example, the same or similar in structure and/or function to any system or device described herein, such as the system 100 described above. For example, as shown in FIG. 3C, the system 300 includes an elongated tubular member 310, an echogenic member implemented as an inflatable member 320), and magnetic members 330. The inflatable member 320 is coupled to the elongated tubular member 310. The magnetic members 330 can be coupled to and/or disposed within the elongated tubular member 310. The system 300 includes an external magnetic assembly 340) and an ultrasound probe 350.

The elongated tubular member 310 can have a first end 311 and a second end 313 and can define a first lumen (not shown) extending from a first port 312A defined in the first end 311 to a second port 312B defined in the second end 313 such that the first lumen can define a fluid path from an internal cavity C of a patient to an area external to the patient. The elongated tubular member 310 can define a second lumen (not shown) extending from a first port 314A defined in a sidewall of the elongated tubular member 310 to a second port 314B defined near the second end 313 of the elongated tubular member 310 such that the second lumen can define a fluid path from the cavity C of a patient to an area external to the patient. The second port 314B can include a fluid connector configured to be coupled to, for example, a source of fluid or a source of negative pressure. The elongated tubular member 310 can also include a coupling member 315 (e.g., disposed near or adjacent to the second end 313 of the elongated tubular member 310) configured to be operatively coupled to an ultrasonic pulse generator such that the elongated tubular member 310 can deliver ultrasonic pulses transmitted by an ultrasonic pulse generator from the first end 311 of the elongated tubular member 310 to a location within the cavity C.

The inflatable member 320 and the magnetic member 330 can be coupled to the elongated tubular member 310 near the first end 311. In some implementations, the magnetic members 330 are arranged linearly. In some implementations, the magnetic members 330 can be arranged in other arrangements, such as in a multi-column 2D or 3D array or circumferentially around a central axis of the elongated tubular member 310. In some implementations, the magnetic members 330 can be disposed proximally of the inflatable member 320. In some implementations, one, some, or all of the magnetic members 330 can be disposed distally of the inflatable member 320 and/or aligned with (e.g., within, coupled to a surface of, or surrounded by) the inflatable member 320.

The first end 311 can be configured to be disposed or introduced through an opening of a patient (e.g., through an incision) and into the cavity C of a patient such that the elongated tubular member 310 can provide access to the cavity C from an exterior of the patient via the first lumen 312. The cavity C can be, for example, a pleural and/or peritoneal space, such as of a chest or abdominal cavity. The cavity C can be defined by a lining L (e.g., a pleural or peritoneal lining) and an organ B (e.g., a bowel or lung). The lining L can include, for example, any tissue, lining, partition, membrane, or wall that is internal to the patient and defines a boundary of the cavity C, as shown in FIG. 3A.

As shown in FIG. 3A, the first end 311 of the elongated tubular member 310 can be introduced to the cavity C via an introducer assembly 370. The introducer assembly 370 can include a hollow needle 372 and a guidewire 374. The introducer assembly 370 can be used to provide access to a cavity C of a patient using, for example, a needle over guidewire technique such as the Seldinger technique. The Seldinger technique can include, for example, using a hollow needle 372 to creating an opening O in a cutaneous surface S of the patient. The hollow needle 372 can be translated through the cutaneous surface S and the lining L of the cavity C to form an access path (i.e., a tract) from an exterior to the patient to the cavity C. The ultrasound probe 350) can be disposed on the cutaneous surface S and used to visualize a portion of the hollow needle 372 within the patient. The guidewire 374 can then be advanced into the cavity C through a lumen of the hollow needle 372. The hollow needle 372 can be withdrawn over the guidewire 374 and removed, leaving the guidewire 374 disposed within the tract formed by the hollow needle 372.

As shown in FIG. 3B, the tract formed by the hollow needle 372 can be dilated via a dilator 376 to prepare the tract to accommodate the diameter of the elongated tubular member 310. A diameter of the dilator 374 can correspond to the diameter of the elongated tubular member 310. The dilator 376 can be passed over the guidewire 374 and into the patient towards the cavity C to dilate the tract. The dilator 376 can then be withdrawn over the guidewire 374 and removed from the patient, leaving the guidewire 374 disposed in the dilated tract.

As shown in FIG. 3C, after removing the dilator 376, the elongated tubular member 310 can be passed over the guidewire 374 and into the patient. For example, an end of the guidewire 374 disposed external to the patient can be threaded through the first port 312A, through the first lumen, and out of the second port 312B. The elongated tubular member 312 can then be advanced relative to the guidewire 374 such that the first end 311 of the elongated tubular member 312 is disposed within the cavity C and the second end 313 is disposed on an opposite side of the cutaneous surface S and the lining L and external to the patient. The inflatable member 320) can be in an uninflated configuration while the first end 311 is translated over the guidewire 374 and into the cavity C.

With the first end 311 and the inflatable member 320 disposed within the cavity C. the inflatable member 320) can be transitioned from the uninflated to an inflated configuration. For example, the inflatable member 320) can be filled with an echogenic fluid (e.g., water or saline) such that an interior of the inflatable member 320 can be visualized using the ultrasound probe 350. The inflatable member 320 can be, for example, a balloon. Alternatively or in addition, in some implementations, the elongated tubular member 310 can be formed of or include an echogenic material. In such implementations, the inflatable member 320 may optionally not be included.

The external magnetic assembly 340 can be applied to the cutaneous surface S of the patient such that a skin-facing surface of the external magnetic assembly 340 contacts the cutaneous surface S. The external magnetic assembly 340 can be applied, for example, to a portion of the cutaneous surface S near or adjacent the location of the first end 311 of the elongated tubular member 310 and/or the inflatable member 320 as visualized by the ultrasound probe 350) such that the external magnetic assembly 340 and the magnetic members 330) can magnetically interact. In response to the external magnetic assembly 340 being disposed on the cutaneous surface S, the magnetic members 330) can be urged (e.g., drawn) toward the external magnetic assembly 340 and the lining L. In response to the magnetic members 330 being urged toward the external magnetic assembly 340 and the lining L, the first end 311 of the elongated tubular member 310 can be urged toward the lining L. In some implementations, the magnetic attraction between the external magnetic assembly 340 and the magnetic members 330 through the cutaneous surface S, the lining L, and any other intervening tissue is sufficiently great such that the inflatable member 311 can be urged into contact with the lining L. The ultrasound probe 350) can visualize the interior of the inflatable member 311 through the cutaneous surface S, the lining L, and any other intervening tissue and the position of the inflatable member 311 (and, thus, the first end 311 of the elongated tubular member 310) can be confirmed.

As indicated by the absence of the guidewire 374 in FIG. 3D, the guidewire 374 (shown in FIG. 3C) can be removed from the patient via withdrawing the guidewire 374 through the first lumen and out of the first port 312A of the elongated tubular member 310, such that the guidewire 374 is completely removed from elongated tubular member 310. The position of the first end 311 of the elongated tubular member 310 can be controlled and/or advanced by moving the external magnetic assembly 340 along the cutaneous surface S. For example, the external magnetic assembly 340) can be translated along the cutaneous surface S while maintaining contact between the skin-contacting surface of the external magnetic assembly 340 and the cutaneous surface S. In response to the external magnetic member 340 being translated along the cutaneous surface S, the magnetic members 330 can be urged in the same direction of translation as the external magnetic member 340) (e.g., toward the external magnetic assembly 340) due to the magnetic attraction of the magnetic members 330 to the external magnetic assembly 340. Thus, the first end 311 can also move in the direction of travel of the external magnetic assembly 340 and the inflatable member 320 can maintain contact with the inner surface of the lining L as the first end 311 translates. In some implementations, the inflatable member 320 can be pulled along the inner surface of the lining L as the external magnetic assembly 340 is moved along the cutaneous surface S. The ultrasound probe 350 can be moved with the external magnetic assembly 340 such that the location of the inflatable member 320 (and thus, the location of the first end 311) can be visually confirmed (e.g., throughout the travel of the first end 311).

In some implementations, as shown in FIG. 3D, the system 300 can be used to perform mechanical lysis of fibrinous septations FS. The fibrinous septations FS may define loculated pockets within the cavity C. The external magnetic assembly 340 can be moved along the cutaneous surface S toward a portion of the cutaneous surface S corresponding to (e.g., adjacent or overlying) the fibrinous septations FS such that the external magnetic assembly 340 urges the magnetic members 330 toward the fibrinous septations FS. The movement of the magnetic members 330 in the direction of the fibrinous septations FS can translate the first end 311 through the fibrinous septations FS and into the loculated pockets such that the boundary of the loculated pockets is disrupted. The ultrasound probe 350 can be used to visualize the inflatable member 320 such that the location of the first end 311 relative to the loculated pockets defined by the fibrinous septations FS can be confirmed. In various implementations, mechanical lysis of fibrinous septations FS is performed in proximity of lining L. Over time, as the organ B (e.g., a lung) expands, deeper pockets in the cavity C defining by the fibrinous septations FS can be pushed closer to the pleural lining L region, thus, allowing for mechanical lysis of these fibrinous septations FS with the system 300.

As shown in FIGS. 3E-3F, a treatment substance 382 (e.g., a medication) can be delivered to the cavity C via the elongated tubular member 310. For example, as shown in FIG. 3E, a source of fluid 380) (also referred to as a “first source of fluid 380”) can be coupled to the second port 312B of the elongated tubular member 310 (e.g., via a fluid connector). Fluid can be delivered from the source of fluid 380 through the second port 312B, the first lumen, and through the first port 312A. The fluid can include the treatment substance 382. The source of fluid 380 can include, for example, a container (e.g., a bottle) or a syringe.

In some implementations, the treatment substance 382 can include a medication for fibrinolysis. For example, as shown in FIG. 3F, the fibrinous septations FS may dissolve as the treatment substance 382 contacts the fibrinous septations FS within the cavity C. In some implementations, the treatment substance 382 can include a tissue plasminogen activator (TPA), deoxyribonuclease (DNAse), an antibiotic, a chemotherapy substance, and/or talc. In some implementations, the treatment substance 382 can alternatively or additionally include any other suitable substance or material for any suitable medical procedure.

In some implementations, the system 300 can be configured to mechanically debride fibrinous septations, such as the fibrinous septations FS shown in FIG. 3G. For example, the system 300 can mechanically debride the fibrinous septations FS via ultrasonic fibrinolysis in addition to delivering the treatment substance 382. In some implementations, an ultrasonic pulse generator 390 can be used in conjunction with the system 300 by interfacing, attachment, and/or coupling with the elongated tubular member 310. For example, the ultrasonic pulse generator 390 can be configured to be coupled to the coupling member 315 (e.g., an adapter) of the elongated tubular member 310. In some implementations, the system 300 can include one or more pulse delivery mechanisms (e.g., one or more piezoelectric transducers) coupled to or near the first end 311 of the elongated tubular member 310 (e.g., embedded within the first end 311). The system 300 can include one or more wires or leads disposed within the elongated tubular member 310 and coupling the one or more pulse delivery mechanisms to the coupling member 315, and, thus, to the ultrasonic pulse generator. For example, the system 300 can include one wire or lead per piezoelectric transducer or one for an array of piezoelectric transducers included in the system 300. The one or more pulse delivery mechanisms can be oriented within the elongated tubular member 310 and/or relative to the elongated tubular member 310 (e.g., oriented such that a pulse generated by the pulse delivery mechanism(s) is transmitted away from a central axis of the elongated tubular member 310 and/or multiple pulses simultaneously delivered by multiple pulse delivery mechanisms are transmitted toward a common region or area such that the pulse delivery is asymmetric relative to the central axis of the elongated tubular member 310) such that manipulation (e.g., translation and/or rotation) of the elongated tubular member 310 can aim the ultrasonic pulses produced by the pulse delivery mechanisms toward a particular area or region. In some implementations, an external magnetic assembly (e.g., the external magnetic assembly 140) can be used to urge the magnetic member(s) 330) via magnetic interaction such that movement of the magnetic member(s) 330) under control of the external magnetic assembly can move (e.g., turn, rotate, and/or translate) a distal portion of the elongated tubular member 310) and cause the pulse delivery mechanism(s) to be oriented to target certain FS with ultrasonic pulses. The ultrasonic pulse generator 390 can be configured to generate and deliver one or more signals or ultrasonic impulses 391 to a location within the patient, such as to a region of the cavity C, by transmission of a signal or ultrasonic impulse through the coupling member 315, for propagation through or along the elongated tubular member 310 (e.g., along the one or more wires or leads), and for delivery within the patient from the first end 311. The one or more ultrasonic impulses 391 can be applied to the region of the cavity C to facilitate fibrinolysis of the fibrinous septations FS. In some implementations, the ultrasonic pulse generator 390 can be configured to operate continuously for a period of time while coupled to the coupling member 315 such that the ultrasonic impulses 391 can be delivered to a region of the cavity C for the period of time. For example, the period of time may be several minutes to several hours (e.g., 3-5 minutes or 3-5 hours).

As shown in FIG. 3H, the elongated tubular member 310 can be used to facilitate an irrigation and drainage process. For example, as shown in FIG. 3H, a source of fluid 385 (also referred to as a “second source of fluid 385”) can be fluidically coupled to the second port 314B of the second lumen (e.g., via any suitable fluid connector) such that fluid can be provided to the cavity C from the source of fluid 385 via the second port 314B, the second lumen, and the first port 314A in the sidewall of the elongated tubular member 310. A source of negative pressure 386 (e.g., a vacuum source) can be coupled to the second port 312B of the first lumen such that fluid can be drawn from the cavity C via the first port 312A in the first end 311 of the elongated tubular member 310, through the first lumen, and through the second port 312B. Thus, the cavity C can be irrigated with fluid from the source of fluid 385 via the first port 314A, and fluid can be drawn toward the source of negative pressure from the cavity C via the first port 312A. In some implementations, the fluid can be conveyed from the source of fluid 385 by a fluid conveying device (not shown) such as a pump (e.g., a peristaltic pump). In some implementations, the source of fluid 385 can include a syringe and/or a fluid bag. In some implementations, the source of negative pressure 386 can include a syringe configured to draw fluid from the cavity C through the first lumen. As fluid is drawn out of the cavity C, the size of the cavity C can reduce such a distance between that the organ B and the lining L decreases.

While the elongated tubular member 310 is shown having a right-angle bend, the elongated tubular member 310 can have any suitable shape, size, and/or configuration. For example, in some implementations, the elongated tubular member 310 can be straight from the first end 311 to the second end 313. In some implementations, the elongated tubular member 310 can be curved or arched from the first end 311 to the second end 313.

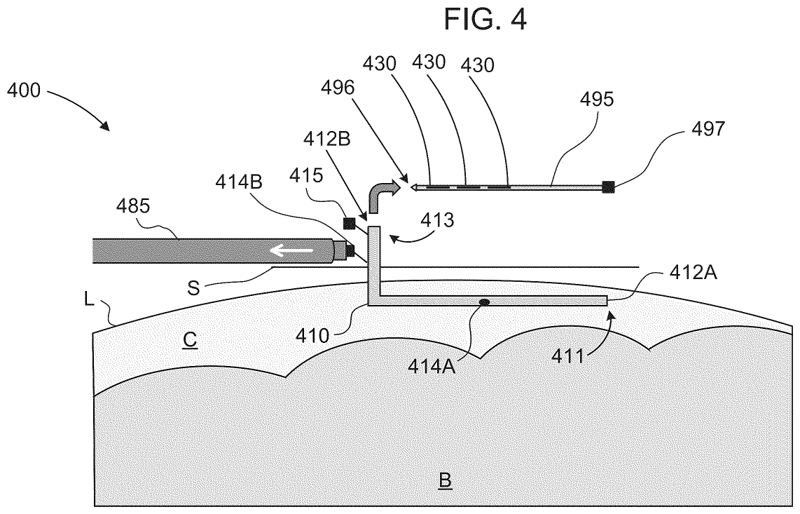

In some embodiments, an elongated tubular member can define a lumen configured to receive an inner sheath and the inner sheath can include or be coupled to magnetic members and define a lumen for fluid communication. For example, FIG. 4 is a schematic illustration depicting a system 400. The system 400 can be the same or similar in structure and/or function to any of the systems described herein, such as the system 100 and/or the system 300 described above. For example, the system 400 includes an elongated tubular member 410) and magnetic members 430. In some implementations, the elongated tubular member 410 can be an indwelling TPC. In some implementations, the elongated tubular member 410 can optionally include an inflatable member (not shown), such as any of the inflatable members described herein (e.g., an echogenic inflatable member). The magnetic members 430 can be coupled to and/or disposed within the elongated tubular member 410. In some implementations, the system 400 can optionally include an external magnetic assembly (not shown) and/or an ultrasound probe (not shown). The elongated tubular member 410 can have a first end 411 and a second end 413 and can define a working channel (e.g., a lumen) extending from an opening 412A (e.g., a first port) defined in the first end 411 to an opening 412B (e.g., a second port) defined in the second end 413. The first end 411 can be configured to be disposed or introduced into a cavity C of a patient, such as along an access path (e.g., a tract) formed through an orifice or incision formed through a cutaneous surface S, any underlying tissue, and a lining L of the patient, such as depicted in FIG. 4. The access path can be formed, for example, using a needle over guidewire technique such as the Seldinger technique, such as described with reference to the system 300 and FIGS. 3A-3B. The cavity C can be, for example, a pleural and/or peritoneal space, such as of a chest or abdominal cavity of the patient. The cavity C can be defined by a lining L (e.g., a pleural or peritoneal lining) and an organ B (e.g., a bowel or lung). The lining L may include, for example, a lining, membrane, wall, or boundary of the cavity C, as shown in FIG. 4.

As shown in FIG. 4, the system 400 includes an inner sheath 495. In some implementations, inner sheath 495 defines a lumen. In some implementations, the lumen of the inner sheath 495 can be a guidewire lumen such that a guidewire disposed within the lumen of the inner sheath 495 can be used to guide the inner sheath 495 and the elongated tubular member 410 through the surface S of the patient and into the cavity C. Alternatively, inner sheath 495 may be a shaft formed as a solid structure without a lumen. For example, the elongated tubular member 410 can define any suitable lumen (e.g., a guidewire lumen) such that a guidewire disposed within the guidewire lumen of the elongated tubular member 410 can be used to guide the elongated tubular member 410 and, optionally, the inner sheath 495, through the surface S of the patient and into the cavity C. The inner sheath 495 can be configured to be removably disposed within the working channel of the elongated tubular member 410. For example, an inner diameter of the working channel can be equal to or greater than an outer diameter of the inner sheath 495 such that the inner sheath 495 can be slidably disposed within the working channel. In some implementations, a length of the inner sheath 495 from a first end 496 to a second end 497 of the inner sheath 495 can be greater than a length of the elongated tubular member 410) from the first end 411 to the second end 413 such that the first end 496 of the inner sheath 495 can protrude from the first end 411 of the elongated tubular member 410 when the second end 497 of the inner sheath 495 is engaged with and/or adjacent to the second end 413 of the elongated tubular member 410.

The elongated tubular member 410 can define a second lumen (not shown) extending from a first port 414A defined in a sidewall of the elongated tubular member 410 to a second port 414B defined near the second end 413 of the elongated tubular member 410 such that the second lumen can define a fluid path from the cavity C of a patient to an area external to the patient. The second port 414B can include a fluid connector configured to be coupled to, for example, a source of fluid or a source of negative pressure.

The inner sheath 495 can include or be coupled to the magnetic members 430. In some implementations, the magnetic member 430 can be coupled to the inner sheath 495 near the first end 496 of the inner sheath 495. Although FIG. 4 shows the inner sheath 495 as including three magnetic members arranged linearly, the inner sheath 495 can include and/or be coupled to any suitable number of magnetic members or any suitable magnetic material in any suitable arrangement. The magnetic members 430 can be the same or similar in structure and/or function to any of the magnetic members described herein, such as, for example, the magnetic members 330 described above.

In some implementations, the elongated tubular member 410 can include a coupling member 415 configured to be coupled to an ultrasonic pulse generator such that the elongated tubular member 410 is configured to deliver one or more ultrasonic impulses from the ultrasonic pulse generator (e.g., from one or more pulse delivery mechanisms disposed at the first end 411 or at a location near the first end) to a region of the cavity C (e.g., to break up fibrinous septations). Alternatively or additionally, in some implementations, the inner sheath 495 can include a coupling member (e.g., near or adjacent to the second end 497 of the inner sheath 495) such that the inner sheath 495 can facilitate propagation of ultrasonic pulses (e.g., generated by an ultrasonic pulse generator coupled to the coupling member) from one or more pulse delivery mechanisms disposed at the first end 496 or at a location near the first end of the inner sheath 495 to a portion of the cavity C.

As shown in FIG. 4, in use, the elongated tubular member 410 can be disposed within a patient to provide access to the cavity C for a period of time. In some implementations, the period of time may be a period of time sufficient to prevent reaccumulation of fluids (e.g., pathologic fluids) within the cavity C. Thus, the cavity C may be continuously or periodically drained while the elongated tubular member 410 is partially disposed within the cavity C. To place the elongated tubular member 410 in a desired position within the cavity C, the inner sheath 495 can be disposed within the working channel of the elongated tubular member 410 (e.g., during insertion of the first end 411 of the elongated tubular member 410 into the cavity C or after the insertion of the first end 411 of the elongated tubular member 410 into the cavity C). An external magnetic assembly, such as any of the external magnetic assemblies described herein, can then be disposed on the cutaneous surface such that the magnetic member 430 of the inner sheath 495 are urged towards the external magnetic assembly. The urging of the inner sheath 495 towards the external magnetic assembly can cause corresponding urging of the elongated tubular member 410 towards the external magnetic assembly and the lining L due to the positioning of the inner sheath 495 within the working channel of the elongated tubular member 410. The external magnetic assembly can then be moved along the cutaneous surface S (e.g., maintaining contact with the cutaneous surface S) to urge the inner sheath 495, and thus the elongated tubular member 410), to a particular location within the cavity C. In some implementations, an ultrasonic probe, such as any of the ultrasonic probes described herein, can be used to visualize the location of a portion of the inner sheath 495 and/or the elongated tubular member 410 (e.g., an echogenic portion) such that the location of the elongated tubular member 410 within the cavity C can be confirmed.

With the first end 411 of the elongated tubular member 410 disposed in a desired location within the cavity C, the inner sheath 495 can be withdrawn relative to the elongated tubular member 410 such that the inner sheath 495 is separated from the elongated tubular member 410, as is shown in FIG. 4. The working channel and/or any lumen of the elongated tubular member 410) can then be used for irrigation and/or drainage of the cavity C. In some implementations, the second port 412B and/or the second port 414B can be coupled to a source of suction to drain or otherwise draw fluids from the cavity C. As shown in FIG. 4, the second port 414B can be coupled to a source of suction 485 to draw fluids from the cavity C, through the first port 414A, through the second lumen, and out of the second port 414B. Thus, the second port 414B can function as a drainage outlet (e.g., with respect to fluids within the cavity C), and the first port 414A is configured to function as a drainage inlet (e.g., with respect to fluids within the cavity C). The source of suction 485 can include, for example, a suction or vacuum pump. In some implementations, the source of suction 485 can include a drainage management system such as a Pleur-evac®.

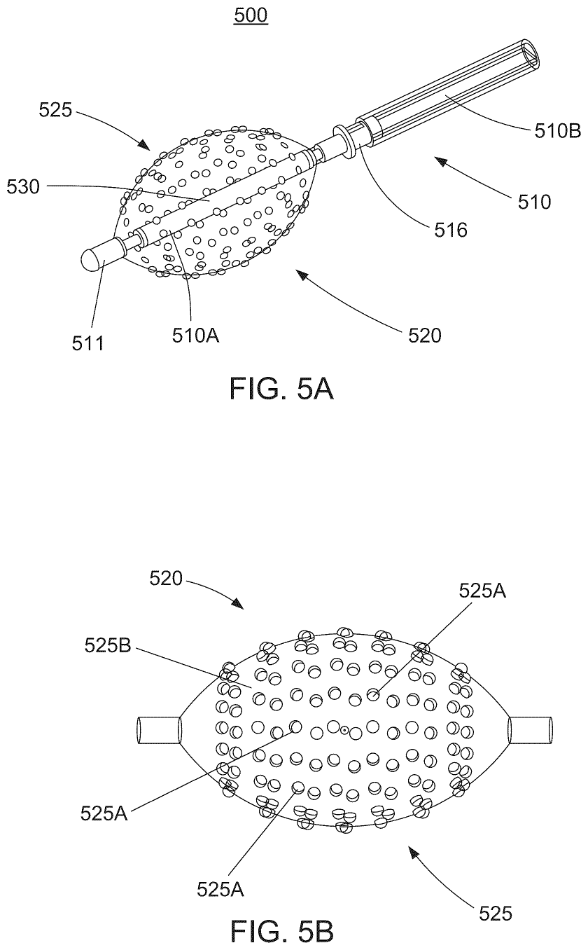

In some embodiments, as described above, an inflatable member can include or be coupled to mechanical abrasion features configured to contact and break down unwanted tissue and/or thick fluid (e.g., for mechanical tissue lysis and/or debridement). For example, FIG. 5A is a schematic illustration of a perspective view of a portion of a system 500. The system 500 can be the same or similar in structure and/or function to of the systems described herein, such as the system 100 and/or the system 300 described above. For example, the system 500 includes an elongated tubular assembly 510, a magnetic member 530, and an inflatable member 520), which may be the same or similar in structure and/or function as any of the elongated tubular members or elongated tubular assemblies, magnetic members, and inflatable members, respectively, described herein, such as the elongated tubular member 110 or 310, the magnetic member 130 or 330, and the inflatable member 120 or 320. Additionally, as shown in FIG. 5A, the system 500 includes a mechanical abrasion feature formed as an array of protrusions 525 disposed on and extending from the outer surface 525B of the inflatable member 520. FIG. 5B is a side view of the inflatable member 520 and the array of protrusions 525.

Some example protrusions 525A of the array of protrusions 525 have been identified in FIG. 5B. As shown in FIGS. 5A and 5B, each protrusion 525A of the array of protrusions 525 can be convex with a rounded surface (e.g., formed as a bump). The protrusions 525A of the array of protrusions 525 can be, for example, hemispherical, or have a greater or smaller radius of curvature than a hemisphere having the same maximum height relative to the outer surface 525B of the inflatable member 520. In some implementations, the protrusions 525A of the array of protrusions 525 can be formed as other shapes or combinations of shapes (e.g., pyramids, cones, ridges, or any other suitable three-dimensional shapes). In some implementations, some or all of the protrusions 525A can have a textured or roughened surface (e.g., compared to the outer surface 525B of the inflatable member 520). In some implementations, some or all of the protrusions 525A can have a smooth surface (e.g., a surface having the same or similar smoothness to the outer surface 525B of the inflatable member 520). In some implementations, the array of protrusions 525 in combination with the outer surface 525B of the inflatable member 520) can form a textured surface due to the protrusions 525A extending away from the outer surface 525B of the inflatable member 520. The array of protrusions 525 can be arranged in any suitable manner on the surface of the inflatable member 520. For example, the protrusions 525A of the array of protrusions 525 can be spaced uniformly over a surface or a portion of a surface of the inflatable member 520 (e.g., in a checkerboard formation or any other suitable formation). In some embodiments, the protrusions 525A of the array of protrusions 525 can be spaced randomly over a surface of the inflatable member 520 such that the distances between adjacent protrusions 525A varies. Any suitable number of protrusions 525A can be included in the array of protrusions 525. For example, in some implementations, the number of protrusions 525A can be between ten and twenty, between twenty and fifty, between fifty and seventy-five, between seventy-five and one hundred, between one hundred and one hundred fifty, between one hundred fifty and two hundred, or between two hundred and three hundred. In use, the array of protrusions 525 can be placed in abrasive contact with unwanted tissue and/or thick fluid and moved (e.g., rotated and/or translated) to break down the unwanted tissue and/or thick fluid using friction between the array of protrusions 525 and the unwanted tissue and/or thick fluid. In some embodiments, the array of protrusions 525 can be used to perform mechanical tissue lysis and/or debridement.

As shown in FIG. 5A, the elongated tubular assembly 510) can include a first tubular portion 510A, a second tubular portion 510B, and a connector 516 mechanically coupling the first tubular portion 510A to the second tubular portion 510B. The elongated tubular assembly 510 has a first end 511 (which may include an end cap coupled to a first or distal end of the inflatable member 520 as shown in FIG. 5A) and a second end opposite the first end (not shown). The distal end 511 can be the same or similar in structure and/or function to the first end 311. In some embodiments, the distal end 511 may be fixedly attached to the inflatable member 520) and/or the first tubular portion 510A. The first tubular portion 510A can be disposed within the inflatable member 520 (e.g., extending from a first end to a second end of the inflatable member 520). Thus, the inflatable member 520 can be coupled to the connector 516 directly or via the first tubular portion 510A. In some embodiments, the connector 516 can be a fluid connector and can fluidically couple at least one lumen defined by or disposed within the first tubular portion 510A to at least one lumen defined by or disposed within the second tubular portion 510B. Thus, the connector 516 can allow fluids to pass from the second tubular portion 510B to the first tubular portion 510A and into the inflatable member 520 to inflate the inflatable member 520 (e.g., via one or more openings in the first tubular portion 510A). Additionally, the connector 516 can allow fluids to pass from the first tubular portion 510A to the second tubular portion 510B such that the inflatable member 520 can be deflated via the elongated tubular assembly 510.

As shown in FIG. 5A, the magnetic member 530 can be disposed within, coupled to, or form a segment or length of the first tubular portion 510B. As described with respect to the magnetic member 130, in some embodiments, the magnetic member 530) and/or one or more optional additional magnetic members can be disposed in any suitable location relative to the inflatable member 520 (e.g., coupled to or disposed within a portion of the elongated tubular assembly 510 outside of the inflatable member 520, coupled to an inner or outer surface of the inflatable member 520). In use, the magnetic member 530) can interact with a magnetic field associated with an external magnetic assembly (such as any of the external magnetic assemblies described herein) such that the external magnetic assembly can urge the magnetic member rotationally and/or translationally to cause rotation and/or translation of the inflatable member 520.

In some implementations, the connector 516 is engaged with the inflatable member 520) and/or the first tubular portion 510A such that the inflatable member 520 can rotate relative to the connector 516 and/or the second tubular portion 510B (and relative to or along with the first tubular portion 510A). For example, as described above, the inflatable member 520 can be rotated via interaction between the magnetic member 530 and an external magnetic assembly. In some implementations, the inflatable member 520 can rotate relative to the connector 516 or a portion of the connector 516. In some implementations, the connector 516 and/or the second tubular portion 510B can be flexible such that the inflatable member 510 and the connector 516 and/or the second tubular portion 510B can be rotated relative to a proximal end of the second tubular portion 510B. For example, the connector 516 and/or the second tubular portion 510B can allow a limited amount of rotation of the inflatable member 520 about the central axis of the connector 516 and/or the second tubular portion 510B under an applied torque (e.g., the connector 516 can allow the inflatable member 520 to twist by a selected angle or a number of revolutions). When the torque is removed, the connector 516 and/or the second tubular portion 510B can automatically reverse the rotation of the inflatable member 520 (e.g., can unwind or untwist) to urge the inflatable member 520 back towards an initial configuration relative to the connector 516 and/or the second tubular portion 510B and to return to an initial state of the connector 516 and/or the second tubular portion 510B from the torqued state. In some implementations, rotation of the inflatable member 520 can cause corresponding rotation of the connector 516 and the entire second tubular portion 510B (e.g., if the proximal end of the second tubular portion 510B is allowed to rotate freely and not immobilized or restrained), and vice versa.

In use, after disposing the distal end 511 of the elongated tubular assembly 510 within a cavity of a patient, the inflatable member 520 can be transitioned from an uninflated to an inflated configuration. For example, the inflatable member 520 can be filled with an echogenic fluid (e.g., water or saline) such that an interior of the inflatable member 520 can be optionally visualized under ultrasound. In the inflated configuration, the array of protrusions 525 can project from the outer surface of the inflatable member 520 and contact any fluid, tissue, or other material surrounding the inflatable member 520. The inflatable member 520 can have a more rigid surface in the inflated configuration than in the uninflated configuration due to increased pressure within the inflatable member 520 such that the array of protrusions 525 can resist any counterpressure due to contact with the fluid, tissue, and/or other material external to the inflatable member 520) without the inflatable member 520 significantly deforming. In some embodiments, the inflatable member 520 can then be optionally advanced and/or retracted relative to thick fluid, tissue, or other material within the cavity such that at least some protrusions 525 A of the array of protrusions 525 frictionally contact and break down the thick fluid, tissue, and/or other material. In some embodiments, the inflatable member 520) can be advanced, retracted, and/or laterally shifted via interaction between the magnetic member 530) an external magnetic assembly and/or via manipulating (e.g., advancing and/or retracting) the elongated tubular assembly 510 from the proximal end disposed outside of the patient. In some embodiments, the inflatable member 520) can alternatively or additionally (e.g., simultaneously or separately) be rotated relative to thick fluid, tissue, or other material within the cavity such that at least some protrusions 525A of the array of protrusions 525 frictionally contact and break down the thick fluid, tissue, and/or other material. In some embodiments, the inflatable member 520) can be rotated in a first and/or second opposite direction via interaction between the magnetic member 530 and an external magnetic assembly and/or via manipulating (e.g., advancing and/or retracting) the elongated tubular assembly 510 from the proximal end disposed outside of the patient. Broken down fluid, tissue, and/or other material can optionally be withdrawn from the cavity through the elongated tubular assembly 510 (e.g., via one or more openings in the distal end 511 or a sidewall of the elongated tubular assembly 510). The inflatable member 520 can be transitioned from the inflated to the uninflated configuration for removal of the inflatable member 520 from the cavity.

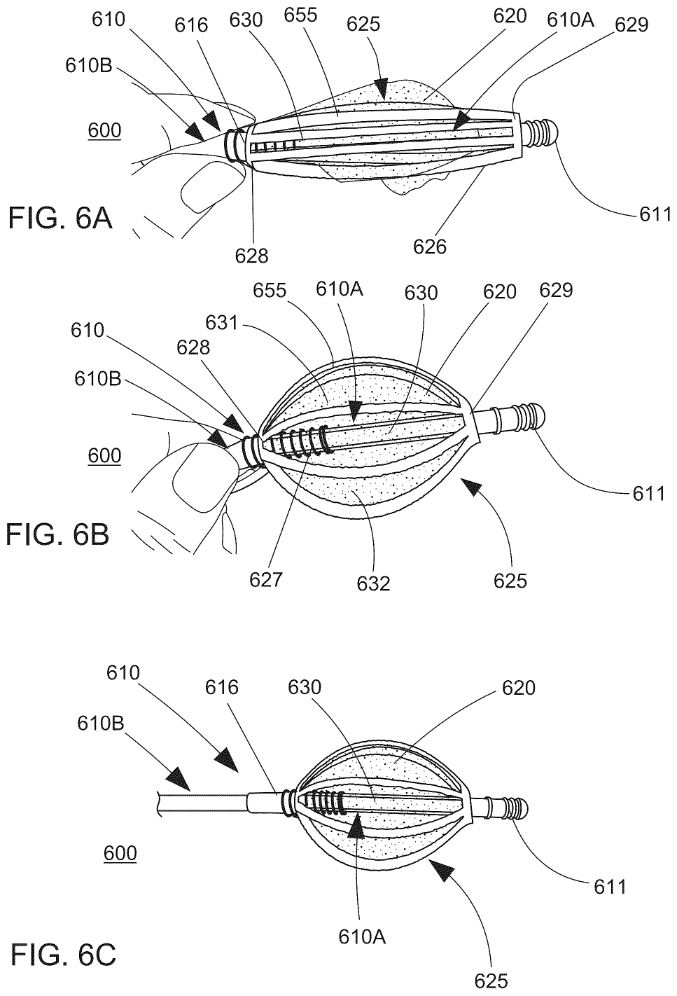

In some embodiments, rather than mechanical abrasion features being formed as an array of protrusions, an inflatable member can include or be coupled to a set of ribs configured to frictionally contact and break down unwanted tissue and/or thick fluid. For example, FIGS. 6A-6C are schematic illustrations of a portion of a system 600. The system 600 can be the same or similar in structure and/or function to of the systems described herein, such as the system 100 and/or the system 300 described above. For example, the system 600 includes an elongated tubular assembly 610, a magnetic member 630, and an inflatable member 620, which may be the same or similar in structure and/or function as any of the elongated tubular members or elongated tubular assemblies, magnetic members, and inflatable members, respectively, described herein, such as the elongated tubular member 110 or 310, the magnetic member 130 or 330, and the inflatable member 120 or 320. Similarly as described with respect to the elongated tubular assembly 510, the elongated tubular assembly 610 can include a first tubular portion 610A, a second tubular portion 610B, and a connector 616 mechanically coupling the first tubular portion 510A to the second tubular portion 510B. The first tubular portion 610A can be disposed within the inflatable member 620, and can be coupled to or include a distal end 611. In some implementations, a spring 627 can be included in the elongated tubular assembly 510 to strengthen the coupling between the connector 616, the first tubular portion 610A, and/or the magnet 630. As shown in FIG. 6A, the magnetic member 630 can be disposed within the first tubular portion 610B and inside the inflatable member 620. Additionally, as shown in FIGS. 6A-6C, the system 600 includes a mechanical abrasion feature formed as set of ribs 625 disposed outside of the inflatable member 620. FIG. 6A shows a side view of the system 600 with the inflatable member 620 and the set of ribs 625 in an initial configuration. FIGS. 6B and 6C show side views of the system 600 with the inflatable member 620 and the set of ribs 625 in an expanded configuration.

Each rib of the set of ribs 625 (e.g., rib 655 identified in FIGS. 6A and 6B) extends from a location at or near a first end of the inflatable member 620 to a location at or near a second end of the inflatable member 620. As shown, the set of ribs 625 can include or be coupled to a first end connector 628 and a second end connector 629. Each rib of the set of ribs 625 can extend from the first end connector 628 to the second end connector 629. The first end connector 628 and the second end connector 629 can each have a circular shape defining a through-hole configured to receive a portion of the elongated tubular member 610. In some implementations, the first end connector 628 can be disposed around or adjacent to the connector 616 of the elongated tubular assembly 610. In some implementations, the second end connector 629 can be disposed around or adjacent to a distal end 611 of the elongated tubular member 610. In some implementations, each rib of the set of ribs 625 can have a textured surface with sufficient roughness such that the set of ribs 625 can be used to break down unwanted tissue and/or thick fluid using frictional contract between the ribs of the set of ribs 625 and the unwanted tissue and/or thick fluid (e.g., while translating and/or rotating the set of ribs 625). In some implementations, for example, each rib of the set of ribs 625 can include roughness elements, such as element 626, as shown in FIG. 6A. The roughness elements 626 may be any suitable protrusions extending from the ribs of the set of ribs 625. The set of ribs 625 can include any suitable number of ribs. For example, although the set of ribs 625 is shown as including eight ribs, in some implementations, the set of ribs 625 can include four, five, six, seven, nine, ten, eleven, twelve, or more ribs. The ribs can be evenly or unevenly spaced about a central axis of the set of ribs 625.

As referenced above, the set of ribs 625 can be transitioned between an unexpanded initial configuration (shown in FIG. 6A) (e.g., for insertion into a body cavity) and an expanded configuration (shown in FIGS. 6B and 6C) (e.g., for frictionally contacting and breaking down fluid and/or tissue). Each rib of the set of ribs 625 can be flexible. In some implementations, each rib of the set of ribs 625 can be disposed in a plane including a central axis of the inflatable member 620) and/or the elongated tubular assembly 610 (e.g., in the unexpanded configuration and in the expanded configuration). In some implementations, in the unexpanded configuration, the ribs of the set of ribs 625 can form substantially straight lines, while in the expanded configuration, the ribs of the set of ribs 625 are curved such that a middle portion of each ribs projects away from a central axis of the elongated tubular assembly 610. In some implementations, in the unexpanded configuration, the ribs of the set of ribs 625 are curved, while in the expanded configuration, the ribs of the set of ribs 625 are more curved than in the unexpanded configuration and thus project farther away from a central axis of the elongated tubular assembly 610. As shown in FIG. 6B, for example, the set of ribs 625 defines larger openings between adjacent ribs (e.g., openings 631 and 632, as shown in FIG. 6B) in the expanded configuration than the unexpanded configuration.

The set of ribs can be transitioned between the unexpanded configuration and the expanded configuration by transitioning the inflatable member 620 disposed within the set of ribs 625 between an initial unexpanded configuration and an expanded configuration (e.g., by inflating the inflatable member 620 from a collapsed uninflated configuration to a substantially spheroidal or spherical inflated configuration). Such a transition may cause the first end connector 628 and/or the second end connector 629 to translate toward each other (e.g., along a portion of the elongated tubular assembly 610).

In use, after disposing the distal end 611 of the elongated tubular assembly 610 within a cavity of a patient, the inflatable member 620 can be transitioned from an uninflated to an inflated configuration to transition the set of ribs 625 between the unexpanded configuration to the expanded configuration. For example, the inflatable member 620 can be filled with an echogenic fluid (e.g., water or saline) such that an interior of the inflatable member 620 can be optionally visualized under ultrasound (e.g., at least through openings defined by the ribs of the set of ribs 625). In some implementations, the set of ribs 625 can be echogenic such that the inflatable member 620 does not need to be echogenic or inflated with an echogenic fluid for visualization of the location of the distal portion of the system 600. In the expanded configuration, the set of ribs 625 can be disposed along and project from the outer surface of the inflatable member 620 and contact any fluid, tissue, or other material surrounding the inflatable member 620. In some embodiments, the inflatable member 620 can then be optionally advanced and/or retracted relative to thick fluid, tissue, or other material within the cavity such that at least some ribs of the set of ribs 625 frictionally contact and break down the thick fluid, tissue, and/or other material. In some embodiments, the set of ribs 625 can be advanced, retracted, and/or laterally shifted via interaction between the magnetic member 630 and an external magnetic assembly and/or via manipulating (e.g., advancing and/or retracting) the elongated tubular assembly 610 from the proximal end disposed outside of the patient. In some embodiments, the set of ribs 625 can alternatively or additionally (e.g., simultaneously or separately) be rotated relative to thick fluid, tissue, or other material within the cavity such that at least some ribs of the set of ribs 625 frictionally contact and break down the thick fluid, tissue, and/or other material. In some embodiments, the set of ribs 625 can be rotated in a first and/or second opposite direction via interaction between the magnetic member 630 and an external magnetic assembly and/or via manipulating (e.g., advancing and/or retracting) the elongated tubular assembly 610 from the proximal end disposed outside of the patient. Broken down fluid, tissue, and/or other material can optionally be withdrawn from the cavity through the elongated tubular assembly 610 (e.g., via one or more openings in the distal end 611 or a sidewall of the elongated tubular assembly 610). The inflatable member 620 can be transitioned from the inflated to the uninflated configuration for removal of the inflatable member 620 and the set of ribs 625 from the cavity. In some embodiments, the set of ribs 625 can be biased toward the unexpanded configuration such that deflation of the inflatable member 620 causes the set of ribs 625 to transition from the expanded to the unexpanded configuration.

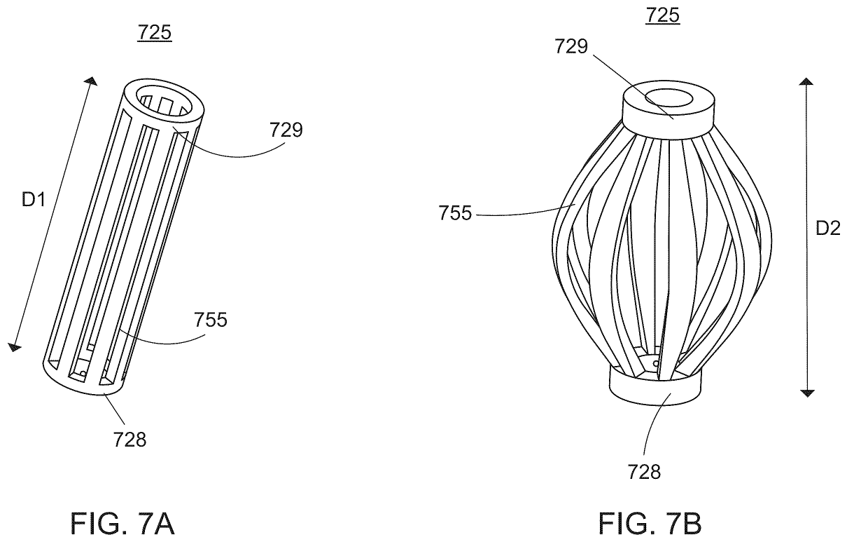

FIGS. 7A and 7B show a three-dimensional schematic view of a set of ribs 725 in an unexpanded configuration and an expanded configuration, respectively. The set of ribs 725 can be the same or similar in structure and/or function as any of the sets of ribs described herein, such as the set of ribs 625. For example, the set of ribs 725 includes a first end connector 728, a second end connector 729, and elongated ribs (e.g., rib 755 identified in FIGS. 7A and 7B) extending from the first end connector 728 to the second end connector 729. As shown in FIG. 7A, the ribs can be substantially straight in the unexpanded configuration. As shown in FIG. 7B, the ribs can be curved in the expanded configuration. In some implementations, in the unexpanded configuration, the distance D1 between the first end connector 728 and the second end connector 729 is larger than distance D2 between the first end connector 728 and the second end connector 729 in the expanded configuration. The set of ribs 725 has a larger maximum diameter (e.g., relative to a central axis of the set of ribs 725 extending through the openings defined by the first end connector 728 and the second end connector 729) in the expanded configuration than in the unexpanded configuration. In some implementations, the ribs of the set of ribs 725 can be biased toward the unexpanded configuration. In some implementations, each rib of the set of ribs 725 can include one or more sharp edges (e.g., extending lengthwise from the first end connector 728 to the second end connector 729) configured to contact and break down unwanted tissue and/or thick fluid. In some implementations, each rib of the set of ribs 725 can have a textured surface with sufficient roughness such that the ribs can be used to break down unwanted tissue and/or thick fluid using frictional contract between the rib and the unwanted tissue and/or thick fluid (e.g., while translating and/or rotating the set of ribs 725). In some implementations, the set of ribs 725 can be magnetic or include magnetic portions such that the set of ribs 725 can be rotated and/or translated under the control of an external magnetic assembly, such as any of the external magnetic assemblies described herein, when disposed within a cavity of a patient. In some implementations, the set of ribs 725 can include or be formed of an echogenic material such that the set of ribs can be visualized using ultrasound.

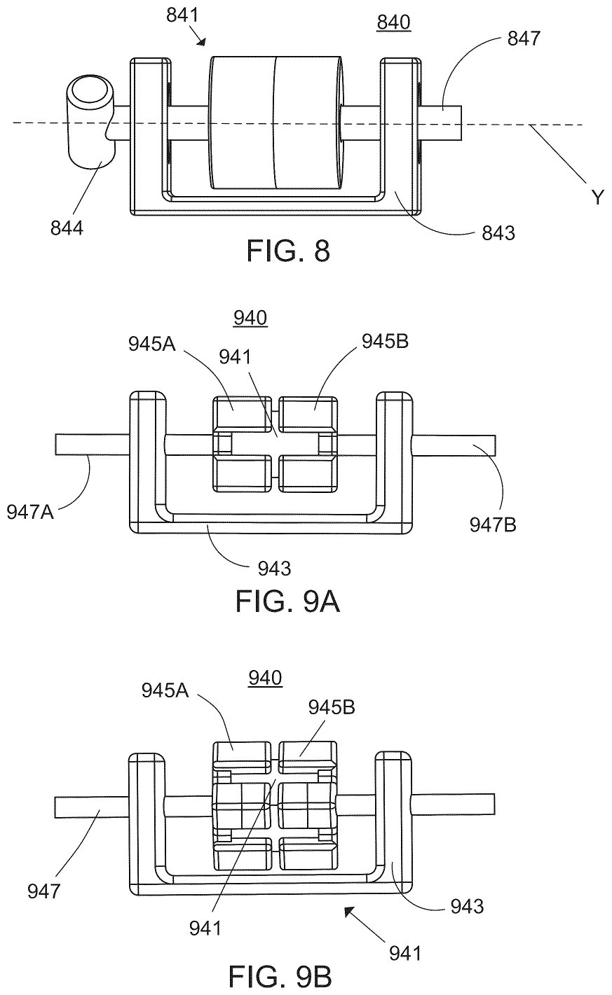

FIG. 8 is a schematic illustration of an external magnetic assembly 840. The external magnetic assembly 840 can be the same or similar in structure and/or function to any of the external magnetic assemblies described herein, such as the external magnetic assembly 140. The external magnetic assembly 840 includes a base 843, an elongated member 847 supported by the base 843, and at least one magnet 841 coupled to and supported by the elongated member 847. In some implementations, for example, the elongated member 847 can extend through a central opening of the at least one magnet 841. The base 843 can support the elongated member 847 near a first end and a second end of the elongated member 847 to maintain the elongated member 847 and the at least one magnet 841 in an elevated position relative to a bottom surface of the base 843. The external magnetic assembly 840 can also include a handle 844 coupled to the elongated member 847 and graspable by a user (e.g., by a hand of the user) to rotate the elongated member 847 and, thus, the at least one magnet 841, about the axis Y (which is coaxial with a central axis of the elongated member 847). In use, the external magnetic assembly 840 can be disposed on a surface of a patient (e.g., on a patient's skin) such that the at least one magnet 841 can magnetically interact with one or more magnetic members disposed in a cavity of the patient (e.g., a magnetic member such as any of the magnetic members described herein). The handle 844 can then be rotated about the axis Y to rotate the at least one magnet 841, causing the one or more magnetic members disposed in the cavity to rotate. Thus, mechanical abrasion feature(s) associated with the one or more magnetic members, such as any of the mechanical abrasion features described herein, can be rotated under control of the external magnetic assembly 840 to break down unwanted tissue and/or thick fluid using frictional contract between the mechanical abrasion features and the unwanted tissue and/or thick fluid. The handle 844 can be continuously rotated to cause corresponding continuous rotation of the mechanical abrasion feature(s). The handle 844 can be rotated in a first direction about the axis Y and/or in a second direction opposite the first direction to cause the at least one magnet 841 to rotated in a first direction and/or a second direction, respectively, In some implementations, the external magnetic assembly can be moved relative to the surface of the patient (e.g., translated along the surface of the skin) to cause the one or more magnetic members in the cavity of the patient to be translated within the cavity.

In some embodiments, rather than an elongated member extending through at least one magnet, an external magnetic assembly can include grips configured to retain the at least one magnet. For example, FIGS. 9A and 9B are schematic illustrations of an external magnetic assembly 940. The external magnetic assembly 940 can be the same or similar in structure and/or function to any of the external magnetic assemblies described herein, such as the external magnetic assembly 140 and/or the external magnetic assembly 840. The external magnetic assembly 940 includes a base 943, a first elongated member 947A and a first grip portion 945A supported by the base 943, a second elongated member 947B and a second grip portion 945B, and at least one magnet 941 coupled to and supported by the first grip portion 945A and the second grip portion 945B. The base 943 can support the at least one magnet 941 on opposite sides of the at least one magnet 941 to maintain the at least one magnet 941 in an elevated position relative to a bottom surface of the base 943. In some implementations, a motor assembly can be operatively coupled to the first elongated member 947A and the second elongated member 947B such that rotation of the at least one magnet 941 can be controlled by the motor assembly (e.g., under the control of a user) in a first and/or second rotational direction. Thus, mechanical abrasion feature(s) associated with the one or more magnetic members, such as any of the mechanical abrasion features described herein, can be rotated (e.g., continuously) under control of the external magnetic assembly 940 to break down unwanted tissue and/or thick fluid using frictional contract between the mechanical abrasion features and the unwanted tissue and/or thick fluid. In some implementations, the motor assembly can include an electric motor powerable by a direct and/or alternating current source. In some embodiments, rather than including or using a motor assembly, the at least one magnet 941 can be rotated manually by a user (e.g., via a handle).

In some implementations, an external magnetic assembly, such as any of the external magnetic assemblies described herein, can include an electromagnet configured to produce a magnetic field upon receipt of an electric current. For example, an external magnetic assembly can include a pulsating electromagnet configured to magnetically interact with magnetic members of a system disposed within a cavity of a patient, such as any of the magnetic members of any of the systems described herein, and to cause the magnetic members to vibrate. The frequency of the vibration of the magnetic members can be controlled, at least in part, based, for example, on an amount of electric current applied to the electromagnet. Vibration of the magnetic members can cause mechanical abrasion feature(s) associated with the magnetic members to vibrate, which can increase abrasive contact between the mechanical abrasion feature(s) and surrounding tissue and/or fluid and improve the break down of the surrounding tissue and/or fluid.

While various embodiments have been described above, it should be understood that they have been presented via example only, and not limitation. Where methods described above indicate certain events occurring in certain order, the ordering of certain events may be modified. Additionally, certain of the events may be performed concurrently in a parallel process when possible, as well as performed sequentially as described above.

Where schematics and/or embodiments described above indicate certain components arranged in certain orientations or positions, the arrangement of components may be modified. While the embodiments have been particularly shown and described, it will be understood that various changes in form and details may be made. Any portion of the apparatus and/or methods described herein may be combined in any combination, except mutually exclusive combinations. The embodiments described herein can include various combinations and/or sub-combinations of the functions, components, and/or features of the different embodiments described.

Moreover, while various embodiments have been described and illustrated herein, those skilled in the art will readily envision, in view of this disclosure, a variety of other means and/or structures for performing the function(s), and/or obtaining the result(s), and/or achieving the advantage(s) described herein, and each of such variations and/or modifications is deemed to be within the scope of the embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented via example only and that, within the scope of the appended claims and equivalents thereto; and that embodiments may be practiced otherwise than as specifically described and claimed without departing from the scope and spirit of the present disclosure. Embodiments of the present disclosure are directed to each individual aspect, feature, system, apparatus, article, material, kit, and/or method described herein. In addition, any combination of two or more of such aspects, features, systems, apparatuses, articles, materials, kits, and/or methods, if such features, systems, apparatuses, articles, materials, kits, and/or methods, are not mutually inconsistent, is included within the inventive scope and spirit of the present disclosure.

Also, various concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Detailed embodiments of the present disclosure are disclosed herein for purposes of describing and illustrating claimed structures and methods that may be embodied in various forms, and are not intended to be exhaustive in any way, or limited to the disclosed embodiments. Many modifications and variations will be apparent to those of ordinary skill in the art without departing from the scope and spirit of the disclosed embodiments. The terminology used herein was chosen to best explain the principles of the one or more embodiments, practical applications, or technical improvements over current technologies, or to enable those of ordinary skill in the art to understand the embodiments disclosed herein. As described, details of well-known features and techniques may be omitted to avoid unnecessarily obscuring the embodiments of the present disclosure.

References in the specification to “one embodiment,” “an embodiment,” “an example embodiment,” or the like, indicate that the embodiment described may include one or more particular features, aspects, implementations, structures, or characteristics, but it shall be understood that such particular features, aspects, implementations, structures, or characteristics may or may not be common to each and every disclosed embodiment of the present disclosure herein. Moreover, such phrases do not necessarily refer to any one particular embodiment per se. As such, when one or more particular features, aspects, implementations, structures, or characteristics is described in connection with an embodiment, it is submitted that it is within the knowledge of those skilled in the art to affect such one or more features, aspects, implementations, structures, or characteristics in connection with one or more other embodiments, where applicable, whether or not explicitly described.

1. A system, comprising:
an elongated tubular member having a first end and a second end, the elongated tubular member defining a first lumen extending from the first end to the second end, the elongated tubular member defining a second lumen extending from a first port defined in a sidewall of the elongated tubular member to a second port in the second end of the elongated tubular member;
an echogenic member coupled to the elongated tubular member;
a magnetic member disposed within the elongated tubular member, a position of the echogenic member and the first end of the elongated tubular member within a cavity of a patient controllable via magnetic attraction between the magnetic member and an external magnetic source disposed external to the patient.

2. The system of claim 1, wherein the elongated tubular member includes a coupling member configured to be operatively coupled to an ultrasonic pulse generator such that the elongated tubular member can deliver ultrasonic pulses transmitted by an ultrasonic pulse generator from the first end of the elongated tubular member to a location within the cavity.

3. The system of claim 1, wherein the echogenic member includes an inflatable member, the inflatable member configured to define an echogenic volume in an inflated configuration.

4.-5. (canceled)

6. The system of claim 1, further comprising a mechanical abrasion feature configured to abrasively contact and break down at least one of a tissue or a thick fluid.

7. The system of claim 6, wherein the mechanical abrasion feature is configured to perform at least one of material lysis or material debridement.

8. The system of claim 6, wherein the mechanical abrasion feature includes an array of projections disposed on and extending from the echogenic member.

9. (canceled)

10. The system of claim 6, wherein the mechanical abrasion feature includes a set of ribs extending along an outer surface of the echogenic member.

11.-16. (canceled)

17. The system of claim 6, wherein the mechanical abrasion feature includes magnetic portions.

18. The system of claim 17, wherein the magnetic portions are arranged such that the mechanical abrasion feature can be rotated within the cavity due to a magnetic interaction with the external magnetic source.

19.-20. (canceled)

21. The system of claim 17, wherein the mechanical abrasion feature is fixed relative to the elongated tubular member and the echogenic member such that a rotation of the mechanical abrasion feature causes a rotation of the echogenic member and at least the first end of the elongated tubular member.

22. A method, comprising:
introducing a first end of an elongated tubular member through an opening of a patient and into a cavity of the patient;
applying an external magnetic assembly to an external surface of the patient such that a magnetic member disposed within the elongated tubular member is urged toward the external magnetic assembly and an echogenic member coupled to the elongated tubular member contacts a surface of a wall of the cavity, the surface of the wall of the cavity and the external surface disposed on opposite sides of at least one tissue surface of the patient;
visualizing the echogenic member within the cavity;
moving the external magnetic assembly along the external surface of the patient such that the first end of the elongated tubular member is advanced within the cavity;
drawing fluid from the cavity through the first end of the elongated tubular member, through a lumen of the elongated tubular member, and out of a second end of the elongated tubular member.

23. (canceled)

24. The method of claim 22, wherein the fluid is a first fluid, further comprising introducing a second fluid to the cavity of the patient via the second end of the elongated tubular member, the lumen of the elongated tubular member, and the first end of the elongated tubular member.

25.-28. (canceled)

29. The method of claim 22, wherein the moving the external magnetic member along the external surface of the patient includes moving the external magnetic assembly such that the first end of the elongated tubular member is advanced to break up fibrinous septations.

30. The method of claim 22, further comprising delivering ultrasonic pulses to a location within the cavity via an ultrasonic pulse generator coupled to a coupling member of the elongated tubular member.

31. (canceled)

32. The method of claim 22, wherein the echogenic member includes an inflatable member coupled to the elongated tubular member, and further comprising inflating the inflatable member with echogenic fluid such that the inflatable member expands from an uninflated configuration to an inflated configuration prior to the visualizing.

33. The method of claim 22, further comprising:
rotating a mechanical abrasion feature coupled to the echogenic member such that the mechanical abrasion feature abrasively contacts and breaks down at least one of a tissue or a thick fluid.

34. The method of claim 33, wherein the rotating is to perform at least one of material lysis or material debridement.

35. The method of claim 33, wherein the mechanical abrasion feature includes an array of projections disposed on and extending from the echogenic member.

36. (canceled)

37. The method of claim 33, wherein the mechanical abrasion feature comprises a set of ribs extending along an outer surface of the echogenic member.

38.-43. (canceled)

44. The method of claim 33, wherein the rotating includes rotating a magnet of the external magnetic assembly such that the mechanical abrasion feature rotates within the cavity of the patient due to a magnetic interaction between the magnetic member rotates and the external magnetic assembly.

45.-48. (canceled)
